(19) United States
(12) Patent Application Publication
(10) Pub. No.: US 2022/0409643 A1
(43) Pub. Date: Dec. 29, 2022

(54) ROR Gamma T Inhibitors and Topical Uses Thereof

(71) Applicant: Dermira, inc., Menlo Park, CA (US)

(72) Inventors: Jamie L. Harden, Menlo Park, CA (US); Hans E.J. Hofland, Menlo Park, CA (US)

(21) Appl. No.: 17/772,815

(22) PCT Filed: Nov. 5, 2020
(86) PCT No.: PCT/US2020/059203

§ 371 Date: Apr. 28, 2022

Related U.S. Application Data
(60) Provisional application No. 62/931,136, filed on Nov. 5, 2019.
(60) Provisional application No. 63/046,154, filed on Jun. 30, 2020.

(51) Int. Cl. A61K 31/695; A61K 9/00; A61K 31/4015; A61K 31/517; A61K 31/513; A61K 31/351; A61K 31/4353; A61K 31/42; A61K 31/4725; A61K 31/4245; A61P 17/06

(52) U.S. Cl. CPC A61K 31/695; A61K 9/0014; A61K 31/4015; A61K 31/517; A61K 31/513; A61K 31/351; A61K 31/4353; A61K 31/42; A61K 31/4725; A61K 31/4245; A61P 17/06

(57) ABSTRACT

The present disclosure is directed to use of RORγt inhibitors in the treatment of autoimmune disorders, e.g., autoimmune disorders of the skin. This disclosure is also directed to pharmaceutical compositions comprising an RORγt inhibitor and a pharmaceutically acceptable carrier for topical administration.

### CROSS-REFERENCE TO RELATED APPLICATIONS

This application claims priority to and the benefit of U.S. Provisional Application Ser. No. 62/931,136, filed on Nov. 5, 2019, and 63/046,154, filed on Jun. 30, 2020, the contents of each of which are hereby incorporated by reference in their entireties.

### BACKGROUND

The retinoic acid receptor-related orphan nuclear receptor (ROR) RORγ and its isoform RORγt play a major role in regulation of a variety of biological systems. RORγt is known to play a central role in immune system development, homeostasis, and responses to microbial pathogens. RORγt is required for the differentiation of Th17 cells, a subset of T helper cells that protect the host from infection by secreting inflammatory cytokines such as IL-17, IL-17A, IL-17F, IL-22, and TNFα. These cytokines are signaling proteins that have been shown to be essential in regulating numerous immune responses, including inflammatory responses to antigens. Th17 cells have also recently been shown to have important roles in activating and directing immune responses in a variety of autoimmune diseases, such as experimental autoimmune encephalomyelitis (EAE), collagen-induced arthritis (CIA), inflammatory bowel disease (IBD), and cancer. Th17 cells have also been implicated in asthma, psoriasis, rheumatoid arthritis, multiple sclerosis, atopic dermatitis and Crohn's disease. Additionally, it has been shown that mice defective for expression of RORγt lack Th17 cells and are resistant to a variety of autoimmune diseases, and that the absence of Th17-inducing microbiota in the small intestine of mice alters the Th17: regulatory T (Treg) cell balance with implications for intestinal immunity, tolerance, and susceptibility to inflammatory bowel diseases.

For example, it has been well established that psoriasis vulgaris is mediated primarily by Th17 polarized T-cells. Biologics targeting the Th17 pathway have proven extremely efficacious in the treatment of this disease. However, biologics are expensive and systemic treatments are typically reserved for patients with severe forms of the disease. RORγt is the master transcription factor for Th17 cell polarization and subsequent Th17 associated cytokine production. RORγt knockout mice are protected against many autoimmune diseases caused by Th17 cells, including psoriasis-like models. Furthermore, pharmacologic blocking RORγt in both murine and human cells and tissues results in inhibition of Th17 polarization and Th17 associated cytokines. Importantly, oral RORγt inhibitors have been tested in humans and found to significantly inhibit IL-17A protein production, demonstrating the role of this key Th17 transcription factor in humans in vivo.

Moderate-severe psoriasis patients are typically administered highly effective biologics, but the mild-moderate psoriasis patient population does not have access to these Th17-specific biologics. First line treatment for mild-moderate patients include topical corticosteroids (TCS), calcipotriol, anthralin, or photochemotherapy, but treat to varying degrees of success and adverse event profiles. Adverse events related to chronic use of steroids make this treatment option less appealing to physicians and mild-moderate patients that do not qualify for biologics, and thus patients often prefer non-steroidal creams. Options such as Vitamin D, while safer, are not as efficacious as topical corticosteroids. Thus, a non-steroidal topical treatment that demonstrates superior or comparable efficacy to TCS that does not carry the same adverse event profile as the known therapeutics on the market is desirable.

### SUMMARY

Described herein are topical compositions comprising RORγt inhibitors and methods for using the RORγt inhibitors for the treatment of autoimmune disorders, such as psoriasis.

Therefore, in a first aspect, the present disclosure provides for a topical composition comprising a RORγt inhibitor, and a pharmaceutically acceptable excipient.

In a second aspect, the present disclosure provides for a method for treating an autoimmune disorder, the method comprising topically administering to a subject in need thereof a topical composition having a therapeutically effective amount of a RORγt inhibitor (e.g. a RORγt inhibitor according to the present disclosure); and a dermatologically acceptable excipient.

In a third aspect, the present disclosure provides a method for reducing inflammation in mammalian skin, the method comprising topically administering to the mammalian skin an effective amount of a topical composition including an RORγt inhibitor (e.g. a RORγt inhibitor according to the present disclosure) and a dermatologically acceptable excipient to a subject in need thereof.

In a fourth aspect, the present disclosure provides a method for reducing the release of an inflammatory biomarker (e.g., IL-17, IL-22), or reducing the incidence of skin lesions on a subject in need thereof, the method comprising topically administering to the mammalian skin a therapeutically effective amount of a topical composition including an RORγt inhibitor (e.g. a RORγt inhibitor according to the present disclosure) to a subject in need thereof.

### DETAILED DESCRIPTION

Provided herein are topical compositions for treating autoimmune disorders, e.g., autoimmune disorders characterized by inflammation. In particular, the pharmaceutical compositions include compounds that are inhibitors of receptor-related orphan nuclear receptor (RORγt). RORγt is the master transcription factor for Th17 cell polarization and subsequent Th17 associated cytokine production. In humans, mutations in Th17 associated genes are highly correlated with autoimmune diseases, including psoriasis. While not wishing to be bound by theory, inhibition of RORγt may attenuate inflammation mediated by Th17, e.g., psoriatic-like skin inflammation.

RORγt Inhibitors for Use in the Compositions and Methods of the Present Disclosure

In one embodiment, the compounds for use in the presently disclosed compositions and/or methods is a compound of Formula I:

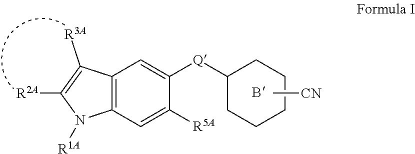

wherein:

R^{1A} is an optionally substituted hydrocarbon group or an optionally substituted hydrocarbon-oxy group;

R^{2A} and R^{3A} are each independently a hydrogen atom, an optionally substituted hydrocarbon group, an optionally substituted hydrocarbon-oxy group, an acyl group, a halogen atom, a cyano group, an optionally substituted hydrocarbon-amino group, an optionally substituted hydrocarbon-sulfanyl group, an optionally substituted hydrocarbon-sulfenyl group, an optionally substituted hydrocarbon-sulfonyl group or a nitro group, or R^{2A} and R^{3A} optionally form, together with the carbon atoms which they are bonded to, an optionally substituted hydrocarbon ring;

R^{SA} is a hydrogen atom or a halogen atom;

Q′ is a bivalent group selected from:

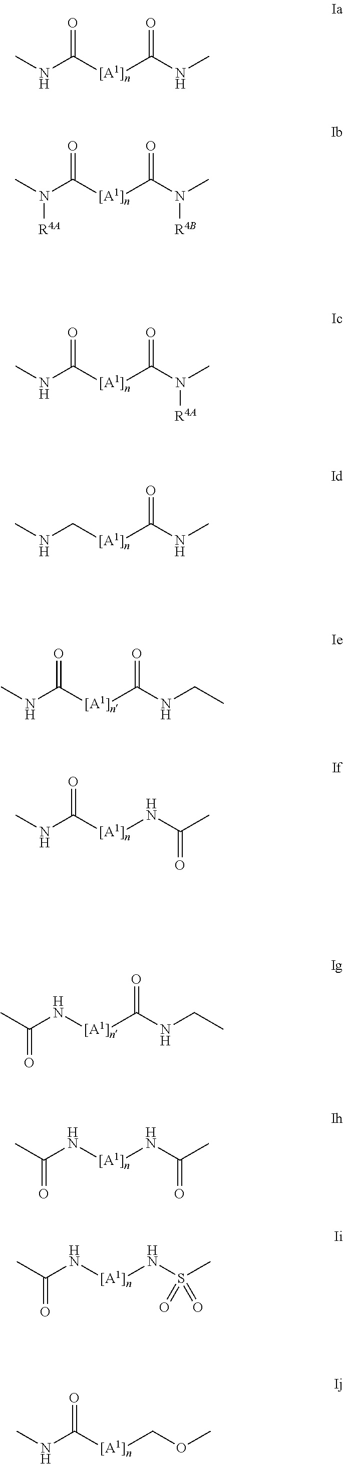

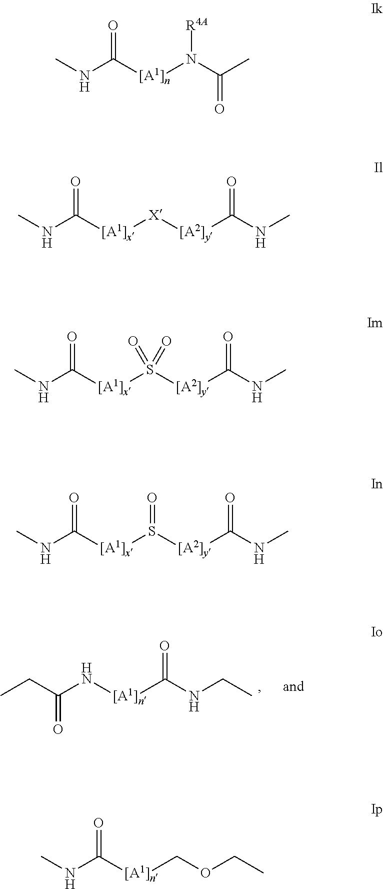

wherein

[A¹] are the same or different and each is a methylene group optionally substituted by substituent(s) selected from a hydroxy group, a phenyl group and an optionally substituted C₁₋₆ alkyl group, wherein the two substituents bonded to the single carbon atom are optionally combined to each other to form a hydrocarbon ring, and [A²] are the same or different and each is a methylene group optionally substituted by substituent(s) selected from a hydroxy group and an optionally substituted C₁₋₆ alkyl group, wherein the two substituents bonded to the single carbon atom are optionally combined to each other to form a hydrocarbon ring, or the methylene group in [A¹] or [A²] is optionally combined to the substituent on the adjacent methylene group to form an optionally substituted hydrocarbon ring,
R^{4A} and R^{4B} are the same or different and each is an optionally substituted hydrocarbon group,
X′ is an oxygen atom, a sulfur atom, or an imino group having an optionally substituted hydrocarbon group or a hydrogen atom,
n is an integer of 1 to 5,
n′ is an integer of 1 to 4,
n″ is an integer of 1 to 3, and
x′ and y′ are each 0 or natural number, and the sum is 0 to 4, and
Ring B′ is a benzene ring optionally having additional substituent(s), or a pyridine ring optionally having additional substituent(s), provided that when R^{SA} is a halogen atom, then Ring B′ is a benzene ring optionally having additional substituent(s), provided that 2-(2-((4-cyanophenyl)amino)-2-oxoethoxy)-N-(9-ethyl-9H-carbazol-3-yl)acetamide and N-(4-cyanophenyl)-N′-(9-ethyl-9H-carbazol-3-yl)-3-methylpentanediamide are excluded) or a stereoisomer, solvates, tautomers, or pharmaceutically acceptable salts thereof.

In another embodiment, the compounds for use in the presently disclosed compositions and/or methods is a compound of Formula II:

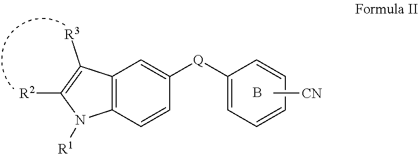

wherein:

R¹ is an optionally substituted hydrocarbon group or an optionally substituted hydrocarbon-oxy group,

R² and R³ are each independently an optionally substituted hydrocarbon group, an optionally substituted hydrocarbon-oxy group, an acyl group, a halogen atom, a cyano group, an optionally substituted hydrocarbon-amino group, an optionally substituted hydrocarbon-sulfanyl group, an optionally substituted hydrocarbon-sulfenyl group, an optionally substituted hydrocarbon-sulfonyl group or a nitro group, or R² and R³ optionally form, together with the carbon atoms which they are bonded to, an optionally substituted hydrocarbon ring,

Q is a bivalent group selected from:

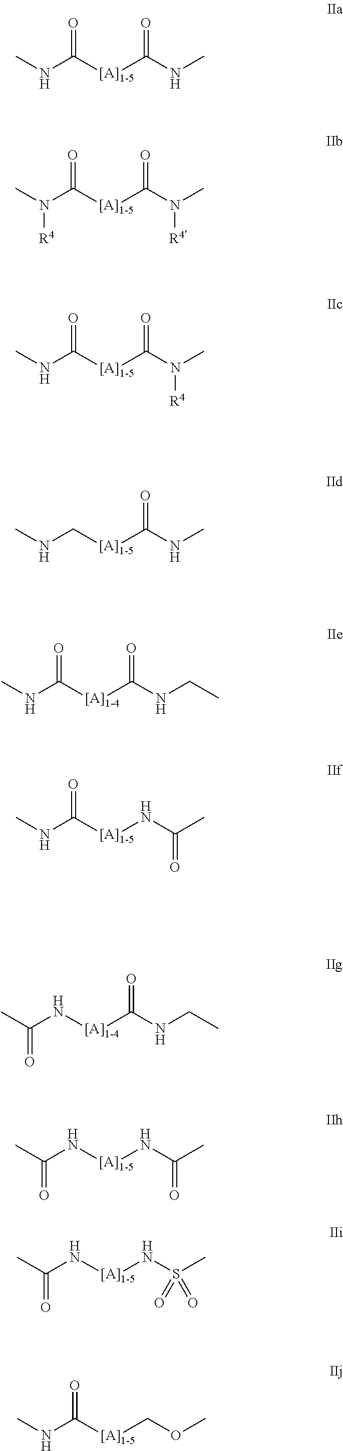

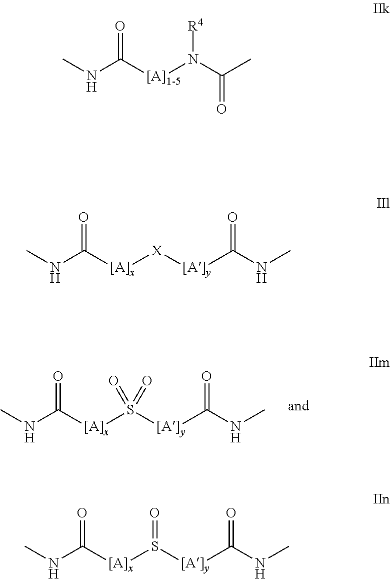

### Wherein:

[A] are the same or different and each is a methylene group optionally substituted by substituent(s) selected from a hydroxy group and an optionally substituted C₁₋₆ alkyl group, wherein the two substituents bonded to the single carbon atom are optionally combined to each other to form a hydrocarbon ring, and [A′] are the same or different and each is a methylene group optionally substituted by substituent(s) selected from a hydroxy group and an optionally substituted C₁₋₆ alkyl group, wherein the two substituents bonded to the single carbon atom are optionally combined to each other to form a hydrocarbon ring, or the methylene group in [A] or [A′] is optionally combined to the substituent on the adjacent methylene group to form an optionally substituted hydrocarbon ring,

R⁴ and R^{4′} are the same or different and each is an optionally substituted hydrocarbon group,

X is an oxygen atom, a sulfur atom, or an imino group having an optionally substituted hydrocarbon group or a hydrogen atom, and

x and y are each 0 or natural number, and the sum is 0 to 4, and

Ring B is a benzene ring optionally further substituted by substituent(s) excluding cyano,

provided that 2-(2-((4-cyanophenyl)amino)-2-oxoethoxy)-N-(9-ethyl-9H-carbazol-3-yl)acetamide and N-(4-cyanophenyl)-N′-(9-ethyl-9H-carbazol-3-yl)-3-methylpentanediamide are excluded (hereinafter sometimes to be referred to as compound (I)),

or a stereoisomer, solvates, tautomers, or pharmaceutically acceptable salts thereof.

Further RORγt inhibitors for use in the methods of and compositions described herein are described in U.S. Pat. No. 9,120,776 B2 and WO 2013/042782 A1, each of which are incorporated herein by reference in their entireties.

In one embodiment, the compounds for use in the presently disclosed compositions and/or methods is a compound of Formula III:

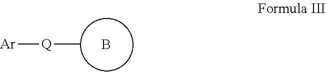

wherein
Ar is the partial structure (1):

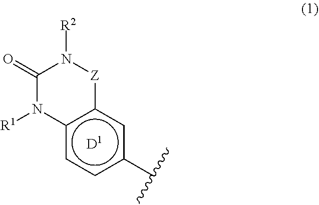

wherein in the partial structure (1),

Z is a carbonyl group or a methylene group,
R¹ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group, an optionally substituted C₇₋₁₆ aralkyl group, an acyl group or a cyano group (excluding a C₁₋₁₂ alkyl group, a C₂₋₁₂ alkenyl group or a C₂-₁₂ alkynyl group, each substituted by optionally substituted

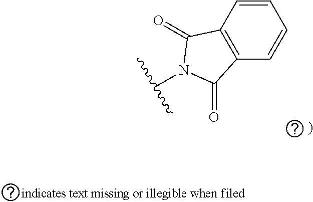 [SMILES: *N1C(=O)c2ccccc2C1=O]

R² is an optionally substituted C₁₋₁₂ alkyl group, an optionally substituted C₂₋₁₂ alkenyl group, an optionally substituted C₂₋₁₂ alkynyl group, an optionally substituted C₃-₁₂ cycloalkyl group, an optionally substituted C₃₋₁₂ cycloalkenyl group, an optionally substituted C₆₋₁₄ aryl group, an optionally substituted C₇₋₁₆ aralkyl group, an acyl group or a cyano group, and

D¹ is an optionally further substituted 6-membered aromatic ring, the partial structure (2):

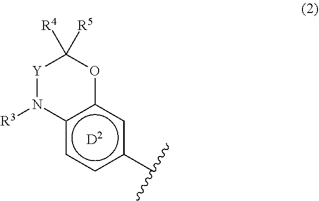

wherein in the partial structure (2),

R³ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group, an optionally substituted C₇₋₁₆ aralkyl group, an acyl group or a cyano group,

Y is an optionally substituted methylene group,

R⁴ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group, an optionally substituted C₇₋₁₆ aralkyl group or an acyl group,

R⁵ is a hydrogen atom or a substituent, or

R⁴ and R⁵ are both methyl groups, or

R⁴ and R⁵ in combination optionally form, together with the carbon atom which they are bonded to, an optionally substituted ring, and

D² is an optionally further substituted 6-membered aromatic ring, or the partial structure (3):

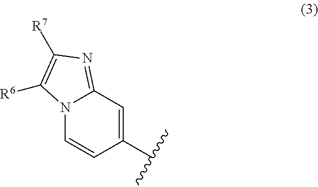

wherein in the partial structure (3),

R⁶ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group or an optionally substituted C₇₋₁₆ aralkyl group, and

R⁷ is an optionally substituted C₁₋₁₂ alkyl group, an optionally substituted C₂-12 alkenyl group, an optionally substituted C₂₋₁₂ alkynyl group, an optionally substituted C₃-12 cycloalkyl group, an optionally substituted C₃-12 cycloalkenyl group, an optionally substituted C₆₋₁₄ aryl group or an optionally substituted C₇₋₁₆ aralkyl group,

Q is a bivalent group selected from the group consisting of the following (Ia)-(Ie):

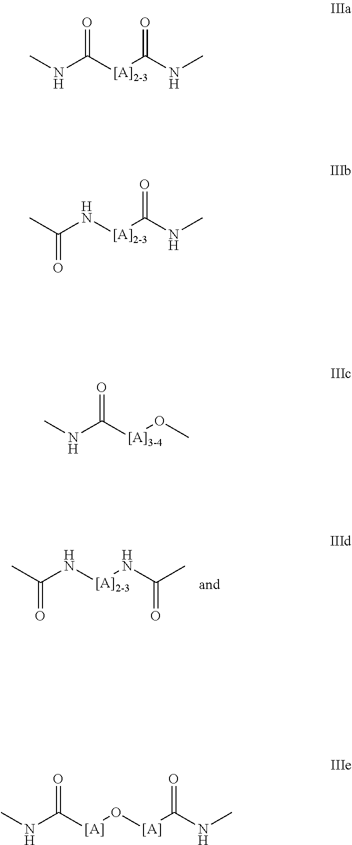

wherein

[A] are the same or different and each is a methylene group optionally substituted by substituent(s) selected from a hydroxy group, an optionally substituted C₁₋₆ alkyl group and a C₆₋₁₄ aryl group, and

B is an optionally substituted ring, provided that
1,2,3,4-tetrahydro-N-[2-[(4-methoxybenzoyl)amino]ethyl]-2,4-dioxo-1-propyl-pyrido[2,3-d]pyrimidine-6-carboxamide, N-[3-[(5-bromo-2-methylphenyl)amino]-3-oxopropyl]-1-cyclopropyl-1,2,3,4-tetrahydro-2,4-dioxo-pyrido[2,3-d]pyrimidine-6-carboxamide, N-[3-[(5-bromo-2-methylphenyl)amino]-3-oxopropyl]-1,2,3,4-tetrahydro-2,4-dioxo-1-propyl-pyrido[2,3-d]pyrimidine-6-carboxamide,

1-cyclopropyl-1,2,3,4-tetrahydro-N-[3-[(6-methyl-2-pyridyl)amino]-3-oxopropyl]-2,4-dioxo-pyrido[2,3-d]pyrimidine-6-carboxamide,

1-cyclopropyl-1,2,3,4-tetrahydro-2,4-dioxo-N-[3-oxo-3-(3-pyridylamino)propyl]-pyrido[2,3-d]pyrimidine-6-carboxamide,

1-cyclopropyl-1,2,3,4-tetrahydro-N-[2-[(2H-indazol-3-ylcarbonyl)amino]ethyl]-2,4-dioxo-pyrido[2,3-d]pyrimidine-6-carboxamide, N-[2-[(2,4-difluorobenzoyl)amino]ethyl]-1,2,3,4-tetrahydro-2,4-dioxo-1-propyl-pyrido[2,3-d]pyrimidine-6-carboxamide,

1,2,3,4-tetrahydro-2,4-dioxo-N-[3-oxo-3-(4-pyridylamino)propyl]-1-propyl-pyrido[2,3-d]pyrimidine-6-carboxamide, N-[2-[(2-chlorobenzoyl)amino]ethyl]-1,2,3,4-tetrahydro-2,4-dioxo-1-propyl-pyrido[2,3-d]pyrimidine-6-carboxamide, N-(2-[(4-chlorobenzoyl)amino]ethyl)-1-cyclopropyl-2,4-dioxo-1,2,3,4-tetrahydropyrido[2,3-d]pyrimidine-6-carboxamide, N-[2-(benzoylamino)ethyl]-1-cyclopropyl-2,4-dioxo-1,2,3,4-tetrahydropyrido[2,3-d]pyrimidine-6-carboxamide, and

1-cyclopropyl-N-[2-[(3-fluoro-4-methylbenzoyl)amino]ethyl]-1,2,3,4-tetrahydro-2,4-dioxo-pyrido[2,3-d]pyrimidine-6-carboxamide are excluded,

or a stereoisomer, solvates, tautomers, or pharmaceutically acceptable salts thereof.

Further RORγt inhibitors for use in the methods of and compositions described herein are described in U.S. Pat. No. 9,834,520 B2 and WO 2014/142255A1, each of which are incorporated herein by reference in their entireties.

In one embodiment, the compounds for use in the presently disclosed compositions and/or methods is a compound of Formula IV:

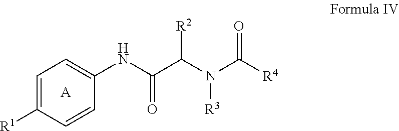

wherein

Ring A is an optionally further substituted 6-membered aromatic ring,

R¹ is
(1) a group represented by the formula: -Q(Ria)(R1b)(R1c) wherein
   Q is a carbon atom, a silicon atom or a germanium atom, and
   R^{1a}, R^{1b} and R^{1c} are each independently a substituent, or
   R^{1a} and R^{1b} in combination optionally form, together with the adjacent Q, an optionally substituted ring,
(2) a neopentyl group, or
(3) a trimethylsilylmethyl group,

R² is
(1) a group represented by the formula:

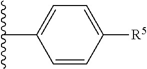

wherein
   R⁵ is an optionally substituted alkyl group or an optionally substituted alkoxy group, and
   the benzene ring in the formula optionally has additional substituent(s) besides R⁵,
(2) an optionally substituted bicyclic fused heterocyclic group, or
(3) a group represented by the formula: -L-Z¹
   wherein
   L is a bond or CH₂, and
   Z¹ is an optionally substituted non-aromatic ring group,

R³ is a hydrogen atom or a substituent, and

R⁴ is a substituent (provided that
(1) a group represented by the formula:

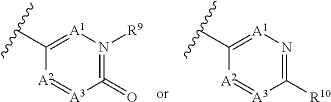

wherein
   A¹ is CR^{A1} wherein R^{A1} is a hydrogen atom or a substituent, or a nitrogen atom,
   A² is CR^{A2} wherein R^{A2} is a hydrogen atom or a substituent, or a nitrogen atom,
   A³ is CR^{A3} wherein R^{A3} is a hydrogen atom or a substituent, or a nitrogen atom, or
   when A² is CR^{A2} wherein R^{A2} is a substituent, and A³ is CR^{A3} wherein R^{A3} is a substituent, then R^{A2} and R^{A3} in combination optionally form, together with the carbon atoms that they are bonded to, a hydrocarbon ring or a heterocycle,
   R⁹ is a hydrogen atom or a hydroxy group, and when R⁹ is a hydroxy group, then A¹, A² and A³ are CR^{A1}, CR^{A2} and CR^{A}, respectively, and
   R¹⁰ is a hydroxy group or an optionally substituted C₁₋₆ alkoxy group, and
(2) an optionally substituted C₁₋₆ alkoxy group are excluded), or
when R³ is a substituent, then R³ and R⁴ in combination optionally form, together with the nitrogen atom adjacent to R³ and the carbon atom adjacent to R⁴, an optionally substituted ring (provided that
   (1) a cyclic group represented by the formula:

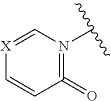

wherein X is CH or a nitrogen atom, which is optionally further substituted, and
(2) a cyclic group represented by the formula:

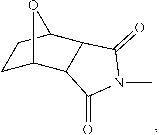

are excluded), and
the substituents that the ring optionally has optionally form a spiro ring, provided that
5-chloro-N-[1-cyclohexyl-2-oxo-2-[[4-[1-(1-pyrrolidinylmethyl)cyclopropyl]phenyl]amino]ethyl]-2-thiophenecarboxamide,
α-(acetylamino)-N-[4-(trifluoromethyl)phenyl]-cyclopentaneacetamide,
α-(acetylamino)-N-[4-(1,1-dimethylethyl)phenyl]-cyclopentaneacetamide,
α-(acetylamino)-N-[2-bromo-4-(trifluoromethyl)phenyl]-cyclopentaneacetamide, and
N-(4-tert-butyl-2-((5-ethyl-2-(2-ethyl-4,4-dimethylpentyl)-7,7-dimethyloctyl)oxy)phenyl)-2-(5,5-dimethyl-2,4-dioxo-1,3-oxazolidin-3-yl)-2-(2-octadecyl-1,1-dioxido-2H-1,2,4-benzthiadiazin-3-yl)acetamide are excluded,
or a stereoisomer, solvates, tautomers, or pharmaceutically acceptable salts thereof.

Further RORγt inhibitors for use in the methods of and compositions described herein are described in US 2017/0107240 A1 and WO 2015/002230 A1, each of which are incorporated herein by reference in their entireties.

In one embodiment, the compounds for use in the presently disclosed compositions and/or methods is a compound of Formula V:

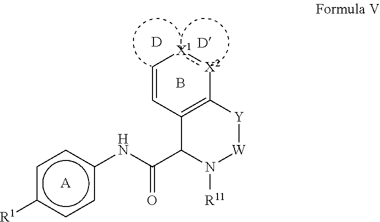

wherein

Ring A is an optionally further substituted 6-membered aromatic ring,

R¹ is
(1) a group represented by the formula: -Q(R^{1a}) (R^{1b}) (R^{1c}) wherein Q is a carbon atom, a silicon atom or a germanium atom, and R^{1a}, R^{1b} and R^{1c} are each independently a substituent, or R^{1a} and R^{1b} in combination optionally form, together with the adjacent Q, an optionally further substituted ring, and R¹c is optionally bonded to one substituent for Ring A to form an optionally further substituted ring,
(2) a neo-pentyl group, or
(3) a trimethylsilylmethyl group,

R¹¹ is —CR¹² R^{12′}R^{12″}, —C(═O)—R⁴ or —SO₂ R¹³,

R¹², R¹² and R^{12′} are each independently a hydrogen atom, a halogen atom, a cyano group, a nitro group, an optionally substituted C₁₋₆ alkyl group, an optionally substituted C₂₋₆ alkenyl group, an optionally substituted C₂₋₆ alkynyl group, an optionally substituted heterocyclic group or an optionally substituted thiocarbamoyl group, R⁴ is an optionally substituted C₁₋₆ alkyl group, an optionally substituted C₂₋₆ alkenyl group, an optionally substituted C₂₋₆ alkynyl group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl(SH) group or an optionally substituted silyl group, wherein the “C₁₋₆ alkyl group”, the “C₂₋₆ alkenyl group” and the “C₂₋₆ alkynyl group” of the “optionally substituted C₁₋₆ alkyl group”, the “optionally substituted C₂₋₆ alkenyl group” and the “optionally substituted C₂₋₆ alkynyl group” for R⁴ are each optionally substituted by 1 to 5 substituents selected from (1) a halogen atom, (2) a nitro group, (3) a cyano group, (4) an oxo group, (5) a hydroxy group, (6) a C₁₋₆ alkoxy group optionally substituted by substituent(s) selected from a halogen atom and a carboxy group, (7) a C₆-₁₄ aryloxy group, (8) a C₇₋₁₆ aralkyloxy group, (9) a 5- to 14-membered aromatic heterocyclyloxy group, (10) a 3- to 14-membered non-aromatic heterocyclyloxy group, (11) a C₁₋₆ alkyl-carbonyloxy group, (12) a C₆₋₁₄ aryl-carbonyloxy group, (13) a C₁₋₆ alkoxy-carbonyloxy group, (14) a mono- or di-C₁₋₆ alkyl-carbamoyloxy group, (15) a C₆-14 aryl-carbamoyloxy group, (16) a 5- to 14-membered aromatic heterocyclylcarbonyloxy group, (17) a 3- to 14-membered non-aromatic heterocyclylcarbonyloxy group, (18) an optionally halogenated C₁₋₆ alkylsulfonyloxy group, (19) a C₆₋₁₄ arylsulfonyloxy group optionally substituted by C₁₋₆ alkyl group(s), (20) an optionally halogenated C₁₋₆ alkylthio group, (21) a 5- to 14-membered aromatic heterocyclic group optionally substituted by substituent(s) selected from a hydroxy group, a C₁₋₆ alkyl group, a C₁₋₆ alkoxy group and a carboxy group, (22) a 3- to 14-membered non-aromatic heterocyclic group optionally substituted by substituent(s) selected from an oxo group and a C₁₋₆ alkyl group, (23) a formyl group, (24) a carboxy group, (25) an optionally halogenated C₁₋₆ alkyl-carbonyl group, (26) a C₆₋₁₄ aryl-carbonyl group, (27) a 5- to 14-membered aromatic heterocyclylcarbonyl group, (28) a 3- to 14-membered non-aromatic heterocyclylcarbonyl group, (29) a C₁₋₆ alkoxy-carbonyl group, (30) a C₆₋₁₄ aryloxy-carbonyl group, (31) a C₇₋₁₆ aralkyloxy-carbonyl group, (32) a carbamoyl group, (33) a thiocarbamoyl group, (34) a mono- or di-C₁₋₆ alkyl-carbamoyl group, (35) a C₆₋₁₄ aryl-carbamoyl group, (36) a 5- to 14-membered aromatic heterocyclylcarbamoyl group, (37) a 3- to 14-membered non-aromatic heterocyclylcarbamoyl group, (38) an optionally halogenated C₁₋₆ alkylsulfonyl group, (39) a C₆₋₁₄ arylsulfonyl group, (40) a 5- to 14-membered aromatic heterocyclylsulfonyl group, (41) an optionally halogenated C₁₋₆ alkylsulfinyl group, (42) a C₆₋₁₄ arylsulfinyl group, (43) a 5—to 14-membered aromatic heterocyclylsulfinyl group, (44) an amino group, (45) a mono- or di-C₁₋₆ alkylamino group (the C₁₋₆ alkyl is optionally substituted by carboxy group(s)), (46) a mono- or di-C₆-14 arylamino group, (47) a 5- to 14-membered aromatic heterocyclylamino group, (48) a C₇₋₁₆ aralkylamino group, (49) a formylamino group, (50) a C₁₋₆ alkyl-carbonylamino group, (51) a (C₁₋₆ alkyl) (C₁₋₆ alkyl-carbonyl)amino group, (52) a C₆₋₁₄ aryl-carbonylamino group, (53) a C₁₋₆ alkoxy-carbonylamino group, (54) a C₇₋₁₆ aralkyloxy-carbonylamino group, (55) a C₁₋₆ alkylsulfonylamino group, (56) a C₆₋₁₄ arylsulfonylamino group optionally substituted by C₁₋₆ alkyl group(s), (57) an optionally halogenated C₁₋₆ alkyl group, (58) a C₂₋₆ alkenyl group, and (59) a C₂₋₆ alkynyl group,

R¹³ is a substituent,

Ring B is a benzene ring, a pyridine ring or a dihydropyridine ring, each of which is optionally further substituted,

the partial structure represented by the formula:

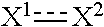

is CR^{5a}═CR⁶, CR^{5b}═N or C(═O)—NR⁷,

R^{5a} and R^{5b} are each independently an optionally substituted alkyl group, an optionally substituted alkoxy group, an optionally substituted alkylsulfonyl group, a cyano group, an optionally substituted cyclic amino group or an oxetan-3-yloxy group, and

R⁶ and R⁷ are each independently a hydrogen atom or a substituent, or the substituent that Ring B optionally further has and R^{5a} or R^{5b} in combination optionally form Ring D, wherein Ring D is a 5- or 6-membered oxygen-containing heterocycle containing 1 to 2 oxygen atoms as heteroatoms in addition to carbon atoms, and is fused at the ring forming position, or

R^{5a} and R⁶ in combination optionally form Ring D′, wherein Ring D′ is a 5- or 6-membered oxygen-containing heterocycle containing 1 to 2 oxygen atoms as heteroatoms in addition to carbon atoms, and is fused at the ring forming position, Y is an optionally substituted methylene group or an oxygen atom, and
W is an optionally substituted C₁₋₂ alkylene group, or a stereoisomer, solvates, tautomers, or pharmaceutically acceptable salts thereof.

In another embodiment, the compounds for use in the presently disclosed compositions and/or methods is a compound of Formula VI:

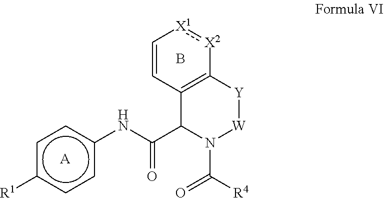

wherein

Ring A is an optionally further substituted 6-membered aromatic ring,

R¹ is
(1) a group represented by the formula: -Q(R^{1a})(R^{1b}) (R^{1c}) wherein Q is a carbon atom, a silicon atom or a germanium atom, and R^{1a}, R^{1b} and R^{1c} are each independently a substituent, or R^{1a} and R^{1b} in combination optionally form, together with the adjacent Q, an optionally further substituted ring, and R^{1c} is optionally bonded to one substituent for Ring A to form an optionally further substituted ring,
(2) a neo-pentyl group, or
(3) a trimethylsilylmethyl group,

R⁴ is a halogen atom, a cyano group, a nitro group, an optionally substituted C₁₋₆ alkyl group, an optionally substituted C₂₋₆ alkenyl group, an optionally substituted C₂₋₆ alkynyl group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl(SH) group or an optionally substituted silyl group,

wherein the “optionally substituted C₁-6 alkyl group”, the “optionally substituted C₂₋₆ alkenyl group” and the “optionally substituted C₂₋₆ alkynyl group” for R⁴ are each optionally substituted by 1 to 5 substituents selected from (1) a halogen atom, (2) a nitro group, (3) a cyano group, (4) an oxo group, (5) a hydroxy group, (6) an optionally halogenated C₁₋₆ alkoxy group, (7) a C₆₋₁₄ aryloxy group, (8) a C₇₋₁₆ aralkyloxy group, (9) a 5- to 14-membered aromatic heterocyclyloxy group, (10) a 3- to 14-membered non-aromatic heterocyclyloxy group, (11) a C₁₋₆ alkyl-carbonyloxy group, (12) a C₆₋₁₄ aryl-carbonyloxy group, (13) a C₁₋₆ alkoxy-carbonyloxy group, (14) a mono- or di-C₁₋₆ alkyl-carbamoyloxy group, (15) a C₆₋₁₄ aryl-carbamoyloxy group, (16) a 5- to 14-membered aromatic heterocyclylcarbonyloxy group, (17) a 3- to 14-membered non-aromatic heterocyclylcarbonyloxy group, (18) an optionally halogenated C₁₋₆ alkylsulfonyloxy group, (19) a C₆₋₁₄ arylsulfonyloxy group optionally substituted by C₁₋₆ alkyl group(s), (20) an optionally halogenated C₁₋₆ alkylthio group, (21) a 5- to 14-membered aromatic heterocyclic group, (22) a 3- to 14-membered non-aromatic heterocyclic group, (23) a formyl group, (24) a carboxy group, (25) an optionally halogenated C₁₋₆ alkyl-carbonyl group, (26) a C₆₋₁₄ aryl-carbonyl group, (27) a 5- to 14-membered aromatic heterocyclylcarbonyl group, (28) a 3- to 14-membered non-aromatic heterocyclylcarbonyl group, (29) a C₁₋₆ alkoxy-carbonyl group, (30) a C₆₋₁₄ aryloxy-carbonyl group, (31) a C₇₋₁₆ aralkyloxy-carbonyl group, (32) a carbamoyl group, (33) a thiocarbamoyl group, (34) a mono- or di-C₁₋₆ alkyl-carbamoyl group, (35) a C₆₋₁₄ aryl-carbamoyl group, (36) a 5- to 14-membered aromatic heterocyclylcarbamoyl group, (37) a 3- to 14-membered non-aromatic heterocyclylcarbamoyl group, (38) an optionally halogenated C₁₋₆ alkylsulfonyl group, (39) a C₆₋₁₄ arylsulfonyl group, (40) a 5- to 14-membered aromatic heterocyclylsulfonyl group, (41) an optionally halogenated C₁₋₆ alkylsulfinyl group, (42) a C₆₋₁₄ arylsulfinyl group, (43) a 5—to 14-membered aromatic heterocyclylsulfinyl group, (44) an amino group, (45) a mono- or di-C₁₋₆ alkylamino group, (46) a mono- or di-C₆₋₁₄ arylamino group, (47) a 5- to 14-membered aromatic heterocyclylamino group, (48) a C₇₋₁₆ aralkylamino group, (49) a formylamino group, (50) a C₁₋₆ alkyl-carbonylamino group, (51) a (C₁₋₆ alkyl) (C₁₋₆ alkyl-carbonyl)amino group, (52) a C₆₋₁₄ aryl-carbonylamino group, (53) a C₁₋₆ alkoxy-carbonylamino group, (54) a C₇₋₁₆ aralkyloxy-carbonylamino group, (55) a C₁₋₆ alkylsulfonylamino group, (56) a C₆₋₁₄ arylsulfonylamino group optionally substituted by C₁₋₆ alkyl group(s), (57) an optionally halogenated C₁₋₆ alkyl group, (58) a C₂₋₆ alkenyl group, and (59) a C₂₋₆ alkynyl group,

Ring B is a benzene ring, a pyridine ring or a dihydropyridine ring, each of which is optionally further substituted,

the partial structure represented by the formula:

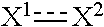

is CR^{5a}═CR⁶, CR^{5b}═N or C(═O)—NR⁷,

R^{5a} and R^{5b} are each independently an optionally substituted alkyl group or an optionally substituted alkoxy group,

R⁶ and R⁷ are each independently a hydrogen atom or a substituent,

Y is an optionally substituted methylene group or an oxygen atom, and

W is an optionally substituted C₁₋₂ alkylene group, or a stereoisomer, solvates, tautomers, or pharmaceutically acceptable salts thereof.

Further RORγt inhibitors for use in the methods of and compositions described herein are described in 10,053,468 B1 and WO 2015/002231 A 1, each of which are incorporated herein by reference in their entireties.

In another embodiment, the compounds for use in the presently disclosed compositions and/or methods is a compound of Formula VII:

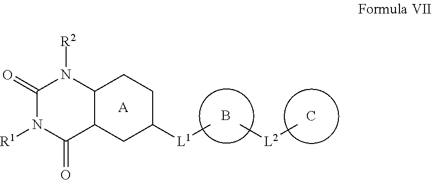

wherein:

R¹ and R² are each independently (1) a methyl group substituted by one substituent selected from (a) an optionally substituted C₃₋₆ cycloalkyl group and (b) an optionally substituted 5- or 6-membered non-aromatic heterocyclic group, (2) an optionally substituted C₂₋₆ alkyl group, or (3) an optionally substituted C₂₋₆ alkenyl group;

ring A is an optionally further substituted 6-membered aromatic ring;

L¹ is a bond, or a spacer having a main chain having 1-3 atoms;
ring B is a non-aromatic ring optionally further substituted by 1 to 3 substituents selected from: (a) an acyl group, (b) an optionally substituted C₁₋₆ alkyl group, (c) an optionally substituted C₁₋₆ alkoxy group, (d) a hydroxy group, (e) a halogen atom and (f) an oxo group;

L² is a bond, or a spacer having a main chain having 1-4 atoms; and

ring C is an optionally further substituted ring,

or a stereoisomer, solvates, tautomers, or pharmaceutically acceptable salts thereof.

In some embodiments, the present disclosure provides a compound according to Formula VII, wherein R¹ and R² are each independently (1) a methyl group substituted by one substituent selected from (a) a C₃₋₆ cycloalkyl group optionally substituted by 1 to 3 halogen atoms and (b) a 5- or 6-membered non-aromatic heterocyclic group, (2) a C₂₋₆ alkyl group optionally substituted by 1 to 3 substituents selected from a halogen atom, a C₁₋₆ alkoxy group and an acyl group, or (3) a C₂₋₆ alkenyl group, or a stereoisomer, solvates, tautomers, or pharmaceutically acceptable salts thereof.

In some embodiments, the present disclosure provides a compound according to Formula VII, wherein L¹ is a bond, or a spacer having a main chain of 1-2 atoms, or a stereoisomer, solvates, tautomers, or pharmaceutically acceptable salts thereof.

In some embodiments, the present disclosure provides a compound according to Formula VII, wherein R² is an optionally substituted C₃-6 alkyl group or an optionally substituted C₃₋₆ alkenyl group, each of which is branched at a carbon atom bonded to a nitrogen atom, or a stereoisomer, solvates, tautomers, or pharmaceutically acceptable salts thereof.

In some embodiments, the present disclosure provides a compound according to Formula VII, wherein R¹ is a C₂₋₆ alkyl group optionally substituted by 1 to 3 substituents selected from (1) a methyl group substituted by one substituent selected from (a) a C₃₋₆ cycloalkyl group optionally substituted by 1 to 3 halogen atoms and (b) a 5- or 6-membered non-aromatic heterocyclic group, or (2) a halogen atom, a C₁₋₆ alkoxy group and a C₁₋₆ alkoxy-carbonyl group; R² is (1) a methyl group substituted by a C₃₋₆ cycloalkyl group, (2) a C₂₋₆ alkyl group optionally substituted by 1 to 3 halogen atoms, or (3) a C₂₋₆ alkenyl group; ring A is (1) a benzene ring optionally further substituted by 1 to 3 halogen atoms, or (2) 6-membered aromatic heterocycle; L¹ is a bond, —C(═O)—, —O—C(═O)—, —CH₂—C(═O)—, —C(═O)—NH—, or —NH—C(═O)—; ring B is C₃₋₁₀ cycloalkane or non-aromatic heterocycle, each of which is optionally further substituted by 1 to 3 substituents selected from (a) an acyl group selected from (i) a carboxy group, (ii) a C₁₋₆ alkyl-carbonyl group optionally substituted by a carboxy group, (iii) a C₁₋₆ alkoxy-carbonyl group optionally substituted by a carboxy group or a C₇₋₁₆ aralkyloxy-carbonyl group, (iv) a C₇₋₁₆ aralkyloxy-carbonyl group, (v) a carbamoyl group and (vi) a C₁₋₆ alkyl-sulfonyl group, (b) a C₁₋₆ alkyl group optionally substituted by a hydroxy group, (c) a hydroxy group and (d) an oxo group; L² is a bond, —O—, —C(═O)—, —CH₂—O—, C(═O)—CH₂—, —C(═O)—NH—optionally substituted by a C₁₋₆ alkyl group optionally substituted by 1 to 3 halogen atoms, —NH—C(═O)—optionally substituted by a C₁₋₆ alkyl group optionally substituted by 1 to 3 halogen atoms, —NH—S(═O)₂—, —CH₂—C(═O)—NH—, —CH₂—NH—C(═O)—, —O—C(═O)—NH—, —NH—C(═O)—NH—, —NH—C(═O)—CH₂—optionally substituted by a hydroxy group, —CH₂—NH—CH₂—optionally substituted by a C₁₋₆ alkyl group optionally substituted by 1 to 3 halogen atoms, —NH—C(═O)—CH₂—CH₂—or —CH₂—NH—C(═O)—NH—; and ring C is a C₆₋₁₄ aromatic hydrocarbon ring, a 5- or 6-membered monocyclic aromatic heterocycle, a 8- to 14-membered fused polycyclic aromatic heterocycle, a 3- to 8-membered monocyclic non-aromatic heterocycle or a 9- to 14-membered fused polycyclic non-aromatic heterocycle, each of which is optionally further substituted by 1 to 3 substituents selected from (1) a cyano group, (2) a hydroxy group, (3) an oxo group, (4) a halogen atom, (5) a C₁₋₆ alkyl group optionally substituted by 1 to 3 substituents selected from a cyano group, a hydroxy group, a halogen atom, a C₁₋₆ alkoxy group, an amino group, a C₁₋₆ alkoxy-carbonylamino group, a C₁₋₆ alkyl-carbonylamino group optionally substituted by a halogen atom, a C₂₋₆ alkenyl-carbonylamino group and a C₁₋₆ alkyl-aminocarbonyloxy group, (6) a C₂₋₆ alkenyl group optionally substituted by a C₁₋₆ alkyl-carbonyl group, (7) a C₃₋₆ cycloalkyl group, (8) a C₆₋₁₄ aryl group, (9) a C₁₋₆ alkoxy group optionally substituted by 1 to 3 substituents selected from a halogen atom and a C₁₋₆ alkoxy group, (10) a C₁₋₆ alkyl-carbonyl group, (11) a carboxy group, (12) a C₂₋₆ alkenyl-carbonyl group, (13) a C₁₋₆ alkoxy-carbonyl group, (14) a carbamoyl group, (15) an amino group, (16) a C₁₋₆ alkyl-carbonylamino group optionally substituted by a halogen atom, (17) a C₁₋₆ alkoxy-carbonylamino group, (18) a C₁₋₆ alkyl-sulfonyl group, (19) a C₂₋₆ alkenyl-carbonylamino group optionally substituted by a mono- or di-C₁₋₆ alkylamino group, (20) a C₂₋₆ alkenyl-sulfonylamino group and (21) a 3- to 8-membered monocyclic non-aromatic heterocycle; or a stereoisomer, solvates, tautomers, or pharmaceutically acceptable salts thereof.

In some embodiments, the present disclosure provides a compound according to Formula VII, wherein R¹ is a methyl group substituted by one substituent selected from (a) a C₃₋₆ cycloalkyl group optionally substituted by 1 to 3 halogen atoms and (b) a 5- or 6-membered non-aromatic heterocyclic group; R² is a C₂₋₆ alkyl group; ring A is a benzene ring optionally further substituted by 1 to 3 halogen atoms; L¹ is —NH—C(═O)—; ring B is a C₃₋₁₀ cycloalkane or a 3—to 8-membered monocyclic non-aromatic heterocycle; L² is a bond, —C(═O)—NH—, —NH—C(═O)—or —NH—C(═O)—NH—; and ring C is a C₆₋₁₄ aromatic hydrocarbon ring, a 5- or 6-membered monocyclic aromatic heterocycle, a 8- to 14-membered fused polycyclic aromatic heterocycle or a 9- to 14-membered fused polycyclic non-aromatic heterocycle, each of which is optionally further substituted by 1 to 3 substituents selected from (1) a cyano group, (2) an oxo group, (3) a halogen atom, (4) a C₁₋₆ alkyl group optionally substituted by 1 to 3 substituents selected from a C₁₋₆ alkoxy-carbonylamino group and a C₁₋₆ alkyl-aminocarbonyloxy group, (5) a C₁₋₆ alkoxy group and (6) a C₁₋₆ alkoxy-carbonyl group; or a stereoisomer, solvates, tautomers, or pharmaceutically acceptable salts thereof.

Further RORγt inhibitors for use in the methods of and compositions described herein are described in U.S. Pat. No. 10,000,488 B1 and WO 2016/039408 A1, each of which are incorporated herein by reference in their entireties.

In further embodiments, the compounds for use in the presently disclosed compositions and/or methods is selected from one or more of the following:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N—methyl-1,2-oxazole-5-carboxamide;
N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)—yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindol-2-yl)-piperidine-1-carboxamide;
(2R)—N-(4-tert-butyl-3-fluorophenyl)-2-(4-(methoxymethyl)phenyl)-2-(((6-oxopyrimidin-1(6H)—yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N—(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N ′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3 S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide;
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)carbamoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)—yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-2-((4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N—methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide;
or a stereoisomer, solvates, tautomers, or pharmaceutically acceptable salts thereof

In several embodiments, the present disclosure provides for a topical composition [Composition 1] comprising a RORγt inhibitor (e.g., a RORγt inhibitor of the present disclosure), and a dermatologically acceptable excipient.

The present disclosure further provides compositions as follows:

1.1 Composition 1, wherein the RORγt inhibitor is a compound according to any of Formulas I, II. III, IV, V, VI, or VII.

1.2 Any of the preceding compositions, wherein the RORγt inhibitor is a compound selected from:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N—methyl-1,2-oxazole-5-carboxamide
N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)—yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-24(4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindol-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N—(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N ′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3 S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide; (3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide;
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)c arb amoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)—yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-24(4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide; or a stereoisomer, solvates, tautomers, or pharmaceutically acceptable salts thereof.

1.3 Any of the preceding compositions, wherein the composition is in the form of a cream, a lotion, a foam, a gel, a spray or an ointment.

1.4 Any of the preceding compositions, wherein the RORγt inhibitor is present at a concentration of about 0.001 wt. % to about 25 wt. %, based on the total weight of the composition.

1.5 Any of the preceding compositions, wherein the RORγt inhibitor is present at a concentration of about 0.1 wt. % to about 5 wt. %, based on the total weight of the composition.

1.6 Any of the preceding compositions, further comprising a skin penetration enhancer, adjuvant, carrier, vehicle (e.g. liposome), solvent, co-solvent, preservatives, viscosity enhancers, pH adjusters, film-forming agents, humectant, glidant, sweetening agent, diluent, preservative, dye/colorant, flavor enhancer, surfactant, wetting agent, dispersing agent, suspending agent, stabilizer, isotonic agent, solvent, emulsifier, anti-oxidant, or chelating agent.

1.7 The preceding composition, wherein the skin penetration enhancer comprises one or more of mannitol, transcutol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.

1.8 The preceding composition, wherein the composition is applied to a patient's skin once daily, every other day, weekly, or monthly.

1.9 The preceding composition, wherein the composition is applied to a patient's skin twice daily.

1.10 The preceding composition, wherein the composition is applied to a patient's skin three times or more daily.

1.11 Any of the preceding compositions, wherein the composition is administered to a patient suffering from an autoimmune disorder.

1.12 The preceding composition, wherein the autoimmune disorder is an autoimmune disorder of the skin.

1.13 The preceding composition, wherein the skin is mammalian skin (e.g., human skin).

1.14 Any of compositions 1.11-1.13, wherein the autoimmune disorder is consequent to dysfunction of Th17 cells or the release of IL-17 (e.g., IL-17A).

1.15 The preceding composition, wherein the autoimmune disorder is psoriasis, dermatitis, alopecia, cutaneous graft-versus-host-disease (cGVHD), ichthyosis, systemic sclerosis, vitiligo, rosacea, acne, urticaria, Behcet's disease, or lupus erythematosus.

1.16 Any of compositions 1.11-1.15, wherein the autoimmune disorder is psoriasis.

1.17 Any of compositions 1.11-1.16, wherein the autoimmune disorder is psoriasis vulgaris (i.e., plaque psoriasis), guttate psoriasis, inverse psoriasis (i.e., flexural psoriasis), pustular psoriasis (e.g., Von Zumbusch psoriasis, palmoplantar pustulosis, acropustulosis), psoriatic arthritis, erythrodermic psoriasis, nail psoriasis or scalp psoriasis.

1.18 Any of compositions 1.11-1.17, wherein the autoimmune disorder is psoriasis vulgaris (i.e., plaque psoriasis).

1.19 Any of compositions 1.10-1.15, wherein the autoimmune disorder is dermatitis.

1.20 Any of compositions 1.10-1.15 or 1.19, wherein the autoimmune disorder is atopic dermatitis (e.g., mild, moderate, or severe (including mild-to-moderate and moderate-to-severe) atopic dermatitis, Asian atopic dermatitis, European atopic dermatitis, pediatric atopic dermatitis), stasis dermatitis, contact dermatitis, dyshidriotic dermatitis, seborrheic dermatitis, neurodermatitis, nummular dermatitis, or stasis dermatitis.

1.21 Any of compositions 1.10-1.15 or 1.19-1.20, wherein the autoimmune disorder is atopic dermatitis (e.g., Asian atopic dermatitis, European atopic dermatitis).

1.22 Any of compositions 1.10-1.15 or 1.19-1.21, wherein the autoimmune disorder is Asian atopic dermatitis.

1.23 Any of compositions 1.11-1.15, wherein the autoimmune disorder is alopecia.

1.24 Any of compositions 1.11-1.15 or 1.23, wherein the autoimmune disorder is alopecia areata (e.g., alopecia totalis, alopecia universalis).

1.25 Any of compositions 1.11-1.15 or 1.23-1.24, wherein the autoimmune disorder is alopecia areata (e.g., alopecia totalis, alopecia universalis), cicatricial alopecia (e.g., frontal fibrosing alopecia, lichen planus (e.g., lichen planopilaris), folliculitis decalvans), telogen effluvium or anagen effluvium.

1.26 Any of compositions 1.11-1.15 or 1.23-1.25, wherein the autoimmune disorder is alopecia areata (e.g., alopecia totalis, alopecia universalis).

1.27 Any of compositions 1.11-1.15, wherein the autoimmune disorder is ichthyosis.

1.28 Any of compositions 1.11-1.15 or 1.27, wherein the autoimmune disorder is acquired ichthyosis, autosomal ichthyosis, autosomal recessive congenital ichthyosis (e.g. CIE or lamellar), Chanarin-Dorfman syndrome, CHILD syndrome, chondrodysplasia punctata syndrome, Darier disease, epidermal nevus syndrome, epidermolytic ichthyosis, erythrokeratodermias, Giroux-Barbeau syndrome, harlequin ichthyosis, Hailey-Hailey disease, ichthyosis en confetti, ichthyosis hystrix, ichthyosis vulgaris, keratosis follicularis spinulosa decalvans, keratitis-ichthyosis-deafness syndrome, multiple sulfatase deficiency, netherton syndrome, pachyonychia congenita, palmoplantar keratoderma, peeling skin syndrome, pityriasis rubra pilaris, Refsum disease, Rud's syndrome, Sjögren-Larsson syndrome, trichiothiodystrophy, or X-linked ichthyosis.

1.29 Any of compositions 1.11-1.15, wherein the autoimmune disorder is systemic sclerosis.

1.30 Any of compositions 1.11-1.15 or 1.29, wherein the autoimmune disorder is morphea, linear scleroderma, limited systemic scleroderma, diffuse systemic scleroderma, CREST syndrome, sine sclerosis.

1.31 Any of compositions 1.11-1.15, wherein the autoimmune disorder is vitiligo.

1.32 Any of compositions 1.11-1.15 or 1.31, wherein the autoimmune disorder is vitiligo (e.g., non-segmental vitiligo or segmental vitiligo).

1.33 Any of compositions 1.11-1.15, wherein the autoimmune disorder is rosacea.

1.34 Any of compositions 1.11-1.15 or 1.33, wherein the autoimmune disorder is erythematotelangiectatic rosacea, papulopustular rosacea (e.g. acne), rhinophyma, or ocular rosacea.

1.35 Any of compositions 1.11-1.15, wherein the autoimmune disorder is uticaria.

1.36 Any of compositions 1.11-1.15 or 1.35, wherein the autoimmune disorder is chronic spontaneous uticaria.

1.37 Any of compositions 1.11-1.15, wherein the autoimmune disorder is Behcet's disease.

1.38 Any of compositions 1.11-1.15, wherein the autoimmune disorder is lupus erythematosus.

1.39 Any of compositions 1.11-1.15 or 1.38, wherein the autoimmune disorder is systemic lupus erythematosus, discoid lupus erythematosus, or drug-induced lupus.

1.40 Any of compositions 1.11-1.15, wherein the autoimmune disorder is cutaneous graft-versus-host-disease (cGVHD).

1.41 Any of the preceding compositions, the subject is a human.

1.42 Any of the preceding compositions, wherein the mammalian skin is human skin.

As used herein, “topical composition” refers to a formulation of a compound of the present disclosure and a medium generally accepted in the art for the delivery of the biologically active compound to mammalian skin, e.g., human skin. Such a medium includes all dermatologically acceptable carriers, diluents or excipients therefor.

“Stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another.

“Solvate” refers to a form of a compound complexed by solvent molecules.

“Tautomers” refers to two molecules that are structural isomers that readily interconvert.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid,

1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The compounds of the present disclosure, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)-for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

“Dermatologically acceptable excipient” includes without limitation any adjuvant, carrier, vehicle, excipient, glidant, sweetening agent, diluent, preservative, dye/colorant, flavor enhancer, surfactant, wetting agent, dispersing agent, suspending agent, stabilizer, isotonic agent, solvent, or emulsifier, including those approved by the United States Food and Drug Administration as being acceptable for dermatological use on humans or domestic animals, or which are known, or are suitable for use in dermatological compositions.

“Optional” or “optionally” means that the subsequently described event of circumstances may or may not occur, and that the description includes instances where said event or circumstance occurs and instances in which it does not. When a functional group is described as “optionally substituted,” and in turn, substituents on the functional group are also “optionally substituted” and so on, for the purposes of this disclosure, such iterations are limited to five, preferably such iterations are limited to two.

Methods of Using the Compounds of the Present disclosure

The Compounds of the Present disclosure are useful in the treatment of autoimmune disorders, e.g., psoriasis. Therefore, administration or use of a preferred RORγt inhibitor as described herein, e.g., a RORγt inhibitor as hereinbefore described, e.g., a Compound of Formula I, II, III, IV, VI, or VII provides a means to regulate the polarization of Th17 cells and the release of inflammatory cytokines (e.g., IL-17A), and in certain embodiments provide a treatment for various autoimmune diseases and disorders.

For example, in one embodiment the present disclosure provides for a method [Method 1] for treating an autoimmune disorder, the method comprising topically administering to a subject in need thereof a topical composition having a therapeutically effective amount of a RORγt inhibitor (e.g. a RORγt inhibitor according to the present disclosure); and a dermatologically acceptable excipient.

The present disclosure further provides further embodiments of Method 1 as follows

1.1 Method 1, wherein the RORγt inhibitor is a compound according to any of Formulas I, II. III, IV, V, VI, or VI.

1.2 Any of the preceding methods, wherein the RORγt inhibitor is a compound selected from:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide
N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)-yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindo1-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N—(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N ′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3 S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide;
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)carbamoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)-yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-2-((4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide; or a stereoisomer, solvates, tautomers, or pharmaceutically acceptable salts thereof.

1.3 Any of the preceding methods, wherein the composition is in the form of a cream, a lotion, a foam, a gel, a spray or an ointment.

1.4 Any of the preceding methods, wherein the RORγt inhibitor is present at a concentration of about 0.001 wt. % to about 25 wt. %, based on the total weight of the composition.

1.5 Any of the preceding methods, wherein the RORγt inhibitor is present at a concentration of about 0.1 wt. % to about 5 wt. %, based on the total weight of the composition.

1.6 Any of the preceding methods, wherein the composition further comprising a skin penetration enhancer, adjuvant, carrier, vehicle (e.g. liposome), solvent, co-solvent, preservatives, viscosity enhancers, pH adjusters, film-forming agents, humectant, glidant, sweetening agent, diluent, preservative, dye/colorant, flavor enhancer, surfactant, wetting agent, dispersing agent, suspending agent, stabilizer, isotonic agent, solvent, emulsifier, anti-oxidant, or chelating agent.

1.7 The preceding method, wherein the skin penetration enhancer comprises one or more of mannitol, transcutol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.

1.8 Any of the preceding methods, wherein the composition is applied to a patient's skin once daily.

1.9 Any of the preceding methods, wherein the composition is applied to a patient's skin twice daily.

1.10 Any of the preceding methods, wherein the composition is applied to a patient's skin three times or more daily.

1.11 Any of the preceding methods, wherein the autoimmune disorder is an autoimmune disorder of the skin.

1.12 The preceding method, wherein the skin is mammalian skin (e.g., human skin).

1.13 Any of the preceding methods, wherein the autoimmune disorder is consequent to dysfunction of Th17 cells or the release of IL-17 (e.g., IL-17A).

1.14 Any of the preceding methods, wherein the autoimmune disorder is psoriasis, dermatitis, alopecia, cutaneous graft-versus-host-disease (cGVHD), ichthyosis, systemic sclerosis, vitiligo, rosacea, acne, urticaria, Behcet's disease, or lupus erythematosus.

1.15 Any of the preceding methods, wherein the autoimmune disorder is psoriasis.

1.16 Any of the preceding methods, wherein the autoimmune disorder is psoriasis vulgaris (i.e., plaque psoriasis), guttate psoriasis, inverse psoriasis (i.e., flexural psoriasis), pustular psoriasis (e.g., Von Zumbusch psoriasis, palmoplantar pustulosis, acropustulosis), psoriatic arthritis, erythrodermic psoriasis, nail psoriasis or scalp psoriasis.

1.17 Any of the preceding methods, wherein the autoimmune disorder is psoriasis vulgaris (i.e., plaque psoriasis).

1.18 Any of methods 1.1-1.14, wherein the autoimmune disorder is dermatitis.

1.19 Any of methods 1.1-1.14 or 1.18, autoimmune disorder is atopic dermatitis (e.g., mild, moderate, or severe (including mild-to-moderate and moderate-to-severe) atopic dermatitis, Asian atopic dermatitis, European atopic dermatitis, pediatric atopic dermatitis), stasis dermatitis, contact dermatitis, dyshidriotic dermatitis, seborrheic dermatitis, neurodermatitis, nummular dermatitis, or stasis dermatitis.

1.20 Any of methods 1.1-1.14 or 1.18-1.19, wherein the autoimmune disorder is atopic dermatitis (e.g., Asian atopic dermatitis, European atopic dermatitis).

1.21 Any of methods 1.1-1.14 or 1.18-1.20, wherein the autoimmune disorder is Asian atopic dermatitis.

1.22 Any of methods 1.1-1.14, wherein the autoimmune disorder is alopecia.

1.23 Any of methods 1.1-1.14 or 1.23, wherein the autoimmune disorder is alopecia areata (e.g., alopecia totalis, alopecia universalis), cicatricial alopecia (e.g., frontal fibrosing alopecia, lichen planus (e.g., lichen planopilaris), folliculitis decalvans), telogen effluvium or anagen effluvium.

1.24 Any of methods 1.1-1.14 or. 1.22-1.23, wherein the autoimmune disorder is alopecia areata (e.g., alopecia totalis, alopecia universalis).

1.25 Any of methods 1.1-1.14, wherein the autoimmune disorder is ichthyosis.

1.26 Any of methods 1.1-1.14 or 1.25, wherein the autoimmune disorder is acquired ichthyosis, autosomal ichthyosis, autosomal recessive congenital ichthyosis (e.g. CIE or lamellar), Chanarin-Dorfman syndrome, CHILD syndrome, chondrodysplasia punctata syndrome, Darier disease, epidermal nevus syndrome, epidermolytic ichthyosis, erythrokeratodermias, Giroux-Barbeau syndrome, harlequin ichthyosis, Hailey-Hailey disease, ichthyosis en confetti, ichthyosis hystrix, ichthyosis vulgaris, keratosis follicularis spinulosa decalvans, keratitis-ichthyosis-deafness syndrome, multiple sulfatase deficiency, netherton syndrome, pachyonychia congenita, palmoplantar keratoderma, peeling skin syndrome, pityriasis rubra pilaris, Refsum disease, Rud's syndrome, Sjögren-Larsson syndrome, trichiothiodystrophy, or X-linked ichthyosis.

1.27 Any of methods 1.1-1.14, wherein the autoimmune disorder is systemic sclerosis.

1.28 Any of methods 1.1-1.14 or 1.27, wherein the autoimmune disorder is morphea, linear scleroderma, limited systemic scleroderma, diffuse systemic scleroderma, CREST syndrome, sine sclerosis.

1.29 Any of methods 1.1-1.14, wherein the autoimmune disorder is vitiligo.

1.30 Any of methods 1.1-1.14 or 1.29, wherein the autoimmune disorder is vitiligo (e.g., non-segmental vitiligo or segmental vitiligo).

1.31 Any of methods 1.1-1.14, wherein the autoimmune disorder is rosacea.

1.32 Any of methods 1.1-1.14 or 1.31, wherein the autoimmune disorder is erythematotelangiectatic rosacea, papulopustular rosacea (e.g. acne), rhinophyma, or ocular rosacea.

1.33 Any of methods 1.1-1.14, wherein the autoimmune disorder is uticaria.

1.34 Any of methods 1.1-1.14 or 1.33, wherein the autoimmune disorder is chronic spontaneous uticaria.

1.35 Any of methods 1.1-1.14, wherein the autoimmune disorder is Behcet's disease.

1.36 Any of methods 1.1-1.14, wherein the autoimmune disorder is lupus erythematosus.

1.37 Any of methods 1.1-1.14 or 1.36, wherein the autoimmune disorder is systemic lupus erythematosus, discoid lupus erythematosus, or drug-induced lupus.

1.38 Any of methods 1.1-1.14, wherein the autoimmune disorder is cutaneous graft-versus-host-disease (cGVHD).

1.39 Any of the preceding methods, the subject is a human.

1.40 Any of the preceding methods, wherein the mammalian skin is human skin.

In another embodiment, the present disclosure provides a method [Method 2] for reducing inflammation in mammalian skin, the method comprising topically administering to the mammalian skin an effective amount of a topical composition including an RORγt inhibitor (e.g. a RORγt inhibitor according to the present disclosure) and a dermatologically acceptable excipient to a subject in need thereof.

2.1 Method 2, wherein the inflammation is consequent to an autoimmune disorder.

2.2 Any of the preceding methods, wherein the RORγt inhibitor is a compound according to any of Formulas I, II. III, IV, V, VI, or VI.

2.3 Any of the preceding methods, wherein the RORγt inhibitor is a compound selected from:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide
N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)-yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindo1-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N—(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N ′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3 S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)—1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide;
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)c arb amoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)-yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-24(4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide; or a stereoisomer, solvates, tautomers, or pharmaceutically acceptable salts thereof.

2.4 Any of the preceding methods, wherein the composition is in the form of a cream, a lotion, a foam, a gel, a spray or an ointment.

2.5 Any of the preceding methods, wherein the RORγt inhibitor is present at a concentration of about 0.001 wt. % to about 25 wt. %, based on the total weight of the composition.

2.6 Any of the preceding methods, wherein the RORγt inhibitor is present at a concentration of about 0.1 wt. % to about 5 wt. %, based on the total weight of the composition.

2.7 Any of the preceding methods, wherein the composition further comprising a skin penetration enhancer, adjuvant, carrier, vehicle (e.g. liposome), solvent, co-solvent, preservatives, viscosity enhancers, pH adjusters, film-forming agents, humectant, glidant, sweetening agent, diluent, preservative, dye/colorant, flavor enhancer, surfactant, wetting agent, dispersing agent, suspending agent, stabilizer, isotonic agent, solvent, emulsifier, anti-oxidant, or chelating agent.

2.8 The preceding method, wherein the skin penetration enhancer comprises one or more of mannitol, transcutol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.

2.9 Any of the preceding methods, wherein the composition is applied to a patient's skin once daily.

2.10 Any of the preceding methods, wherein the composition is applied to a patient's skin twice daily.

2.11 Any of the preceding methods, wherein the composition is applied to a patient's skin three times three times daily.

2.12 Any of methods 2.1-2.11, wherein the autoimmune disorder is an autoimmune disorder of the skin.

2.13 The preceding method, wherein the skin is mammalian skin (e.g., human skin).

2.14 Any of methods 2.1-2.13, wherein the autoimmune disorder is consequent to dysfunction of Th17 cells or the release of IL-17 (e.g., IL-17A).

2.15 Any of methods 2.1-2.14, wherein the autoimmune disorder is psoriasis, dermatitis, alopecia, cutaneous graft-versus-host-disease (cGVHD), ichthyosis, systemic sclerosis, vitiligo, rosacea, acne, urticaria, Behcet's disease, or lupus erythematosus.

2.16 Any of methods 2.1-2.15, wherein the autoimmune disorder is psoriasis.

2.17 Any of methods 2.1-2.16, wherein the autoimmune disorder is psoriasis vulgaris (i.e., plaque psoriasis), guttate psoriasis, inverse psoriasis (i.e., flexural psoriasis), pustular psoriasis (e.g., Von Zumbusch psoriasis, palmoplantar pustulosis, acropustulosis), psoriatic arthritis, erythrodermic psoriasis, nail psoriasis or scalp psoriasis.

2.18 Any of methods 2.1-2.17, wherein the autoimmune disorder is psoriasis vulgaris (i.e., plaque psoriasis).

2.19 Any of methods 2.1-2.15, wherein the autoimmune disorder is dermatitis.

2.20 Any of methods 2.1-2.15 or 2.19, wherein the autoimmune disorder is atopic dermatitis (e.g., mild, moderate, or severe (including mild-to-moderate and moderate-to-severe) atopic dermatitis, Asian atopic dermatitis, European atopic dermatitis, pediatric atopic dermatitis), stasis dermatitis, contact dermatitis, dyshidriotic dermatitis, seborrheic dermatitis, neurodermatitis, nummular dermatitis, or stasis dermatitis.

2.21 Any of methods 2.1-2.15 or 2.19-2.20, wherein the autoimmune disorder is atopic dermatitis (e.g., Asian atopic dermatitis, European atopic dermatitis).

2.22 Any of methods 2.1-2.15 or 2.19-2.21, wherein the autoimmune disorder is Asian atopic dermatitis.

2.23 Any of methods 2.1-2.15, wherein the autoimmune disorder is alopecia.

2.24 Any of methods 2.1-2.15 or 2.23, wherein the autoimmune disorder is alopecia areata (e.g., alopecia totalis, alopecia universalis), cicatricial alopecia (e.g., frontal fibrosing alopecia, lichen planus (e.g., lichen planopilaris), folliculitis decalvans), telogen effluvium or anagen effluvium.

2.25 Any of methods 2.1-2.15 or 2.23-2.24, wherein the autoimmune disorder is alopecia areata (e.g., alopecia totalis, alopecia universalis).

2.26 Any of methods 2.1-2.15, wherein the autoimmune disorder is ichthyosis.

2.27 Any of methods 2.1-2.15 or 2.26, wherein the autoimmune disorder is acquired ichthyosis, autosomal ichthyosis, autosomal recessive congenital ichthyosis (e.g. CIE or lamellar), Chanarin-Dorfman syndrome, CHILD syndrome, chondrodysplasia punctata syndrome, Darier disease, epidermal nevus syndrome, epidermolytic ichthyosis, erythrokeratodermias, Giroux-Barbeau syndrome, harlequin ichthyosis, Hailey-Hailey disease, ichthyosis en confetti, ichthyosis hystrix, ichthyosis vulgaris, keratosis follicularis spinulosa decalvans, keratitis-ichthyosis-deafness syndrome, multiple sulfatase deficiency, netherton syndrome, pachyonychia congenita, palmoplantar keratoderma, peeling skin syndrome, pityriasis rubra pilaris, Refsum disease, Rud's syndrome, Sjögren-Larsson syndrome, trichiothiodystrophy, or X-linked ichthyosis.

2.28 Any of methods 2.1-2.15, wherein the autoimmune disorder is systemic sclerosis.

2.29 Any of methods 2.1-2.15 or 2.28, wherein the autoimmune disorder is morphea, linear scleroderma, limited systemic scleroderma, diffuse systemic scleroderma, CREST syndrome, sine sclerosis.

2.30 Any of methods 2.1-2.15, wherein the autoimmune disorder is vitiligo.

2.31 Any of methods 2.1-2.15 or 2.30, wherein the autoimmune disorder is vitiligo (e.g., non-segmental vitiligo or segmental vitiligo).

2.32 Any of methods 2.1-2.15, wherein the autoimmune disorder is rosacea.

2.33 Any of methods 2.1-2.15 or 2.32, wherein the autoimmune disorder is erythematotelangiectatic rosacea, papulopustular rosacea (e.g. acne), rhinophyma, or ocular rosacea.

2.34 Any of methods 2.1-2.15, wherein the autoimmune disorder is uticaria.

2.35 Any of methods 2.1-2.15 or 2.34, wherein the autoimmune disorder is chronic spontaneous uticaria.

2.36 Any of methods 2.1-2.15, wherein the autoimmune disorder is Behcet's disease.

2.37 Any of methods 2.1-2.15, wherein the autoimmune disorder is lupus erythematosus.

2.38 Any of methods 2.1-2.15 or 2.37, wherein the autoimmune disorder is systemic lupus erythematosus, discoid lupus erythematosus, or drug-induced lupus.

2.39 Any of methods 2.1-2.15, wherein the autoimmune disorder is cutaneous graft-versus-host-disease (cGVHD).

2.40 Any of the preceding methods, the subject is a human.

2.41 Any of the preceding methods, wherein the mammalian skin is human skin.

A further embodiment provides a method [Method 3] for reducing the release of an inflammatory biomarker (e.g., IL-17, IL-22), reducing the incidence of skin lesions on a subject in need thereof, reducing the PASI score of a subject, or reduction of psoriatic plaque in a subject in need thereof, the method comprising topically administering to the mammalian skin a therapeutically effective amount of a topical composition including an RORγt inhibitor (e.g. a RORγt inhibitor according to the present disclosure) to a subject in need thereof.

3.1 Method 3, wherein the inflammatory biomarker is one or more of IL-17 (e.g., IL-17A, IL-17F), IL-21, IL-22, TNFα, and CCL20.

3.2 Any of the preceding methods, wherein the inflammatory biomarker is IL-17 (e.g., IL-17A, IL-17F).

3.3 Any of the preceding methods, wherein the inflammatory biomarker is IL-17A.

3.4 Any of the preceding methods, wherein the incidence of skin lesions is reduced for a sustained period of time.

3.5 Any of the preceding methods, wherein the incidence of skin lesions is reduced for a period of 6 months.

3.6 Any of the preceding methods, wherein the incidence of skin lesions is reduced for a period of 12 months or longer.

3.7 Any of the preceding methods, wherein the RORγt inhibitor is a compound according to any of Formulas I, II. III, IV, V, VI, or VI.

3.8 Any of the preceding methods, wherein the RORγt inhibitor is a compound selected from:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide
N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)-yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindo1-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N-(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide;
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)carbamoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)-yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-2((4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide; or a stereoisomer, solvates, tautomers, or pharmaceutically acceptable salts thereof.

3.9 Any of the preceding methods, wherein the composition is in the form of a cream, a lotion, a foam, a gel, a spray or an ointment.

3.10 Any of the preceding methods, wherein the RORγt inhibitor is present at a concentration of about 0.001 wt. % to about 25 wt. %, based on the total weight of the composition.

3.11 Any of the preceding methods, wherein the RORγt inhibitor is present at a concentration of about 0.1 wt. % to about 5 wt. %, based on the total weight of the composition.

3.12 Any of the preceding methods, further comprising a skin penetration enhancer, adjuvant, carrier, vehicle (e.g. liposome), solvent, co-solvent, preservatives, viscosity enhancers, pH adjusters, film-forming agents, humectant, glidant, sweetening agent, diluent, preservative, dye/colorant, flavor enhancer, surfactant, wetting agent, dispersing agent, suspending agent, stabilizer, isotonic agent, solvent, emulsifier, anti-oxidant, or chelating agent.

3.13 The preceding method, wherein the skin penetration enhancer comprises one or more of mannitol, transcutol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.

3.14 Any of the preceding methods, wherein the composition is applied to a patient's skin once daily.

3.15 Any of the preceding methods, wherein the composition is applied to a patient's skin twice daily.

3.16 Any of the preceding methods, wherein the composition is applied to a patient's skin three times or more daily.

3.17 Any of the preceding methods, wherein the composition is administered to a patient suffering from an autoimmune disorder.

3.18 The preceding method, wherein the autoimmune disorder is an autoimmune disorder of the skin.

3.19 The preceding method, wherein the skin is mammalian skin (e.g., human skin).

3.20 Any of methods 3.17-3.19, wherein the autoimmune disorder is consequent to dysfunction of Th17 cells or the release of IL-17 (e.g., IL-17A).

3.21 The preceding method, wherein the autoimmune disorder is psoriasis, dermatitis, alopecia, cutaneous graft-versus-host-disease (cGVHD), ichthyosis, systemic sclerosis, vitiligo, rosacea, acne, urticaria, Behcet's disease, or lupus erythematosus.

3.22 Any of methods 3.17-3.21, wherein the autoimmune disorder is psoriasis.

3.23 Any of methods 3.17-3.22, wherein the autoimmune disorder is psoriasis vulgaris (i.e., plaque psoriasis), guttate psoriasis, inverse psoriasis (i.e., flexural psoriasis), pustular psoriasis (e.g., Von Zumbusch psoriasis, palmoplantar pustulosis, acropustulosis), psoriatic arthritis, erythrodermic psoriasis, nail psoriasis or scalp psoriasis.

3.24 Any of methods 3.17-3.23, wherein the autoimmune disorder is psoriasis vulgaris (i.e., plaque psoriasis).

3.25 Any of methods 3.17-3.19, wherein the autoimmune disorder is dermatitis.

3.26 Any of methods 3.17-3.23 or 3.25, wherein the autoimmune disorder is atopic dermatitis (e.g., mild, moderate, or severe (including mild-to-moderate and moderate-to-severe) atopic dermatitis, Asian atopic dermatitis, European atopic dermatitis, pediatric atopic dermatitis), stasis dermatitis, contact dermatitis, dyshidriotic dermatitis, seborrheic dermatitis, neurodermatitis, nummular dermatitis, or stasis dermatitis.

3.27 Any of methods 3.17-3.23 or 3.25-3.26, wherein the autoimmune disorder is atopic dermatitis (e.g., Asian atopic dermatitis, European atopic dermatitis).

3.28 Any of methods 3.17-3.23 or 3.25-3.27, wherein the autoimmune disorder is Asian atopic dermatitis.

3.29 Any of methods 3.17-3.21, wherein the autoimmune disorder is alopecia.

3.30 Any of methods 3.17-3.21 or 3.29, wherein the autoimmune disorder is alopecia areata (e.g., alopecia totalis, alopecia universalis), cicatricial alopecia (e.g., frontal fibrosing alopecia, lichen planus (e.g., lichen planopilaris), folliculitis decalvans), telogen effluvium or anagen effluvium.

3.31 Any of methods 3.17-3.21 or 3.29-3.30, wherein the autoimmune disorder is alopecia areata (e.g., alopecia totalis, alopecia universalis).

3.32 Any of methods 3.17-3.21, wherein the autoimmune disorder is ichthyosis.

3.33 Any of methods 3.17-3.21 or 3.32, wherein the autoimmune disorder is acquired ichthyosis, autosomal ichthyosis, autosomal recessive congenital ichthyosis (e.g. CIE or lamellar), Chanarin-Dorfman syndrome, CHILD syndrome, chondrodysplasia punctata syndrome, Darier disease, epidermal nevus syndrome, epidermolytic ichthyosis, erythrokeratodermias, Giroux-Barbeau syndrome, harlequin ichthyosis, Hailey-Hailey disease, ichthyosis en confetti, ichthyosis hystrix, ichthyosis vulgaris, keratosis follicularis spinulosa decalvans, keratitis-ichthyosis-deafness syndrome, multiple sulfatase deficiency, netherton syndrome, pachyonychia congenita, palmoplantar keratoderma, peeling skin syndrome, pityriasis rubra pilaris, Refsum disease, Rud's syndrome, Sjögren-Larsson syndrome, trichiothiodystrophy, or X-linked ichthyosis.

3.34 Any of methods 3.17-3.21, wherein the autoimmune disorder is systemic sclerosis.

3.35 Any of methods 3.17-3.21 or 3.34, wherein the autoimmune disorder is morphea, linear scleroderma, limited systemic scleroderma, diffuse systemic scleroderma, CREST syndrome, sine sclerosis.

3.36 Any of methods 3.17-3.21, wherein the autoimmune disorder is vitiligo.

3.37 Any of methods 3.17-3.21 or 3.36, wherein the autoimmune disorder is vitiligo (e.g., non-segmental vitiligo or segmental vitiligo).

3.38 Any of methods 3.17-3.21, wherein the autoimmune disorder is rosacea.

3.39 Any of methods 3.17-3.21 or 3.38, wherein the autoimmune disorder is erythematotelangiectatic rosacea, papulopustular rosacea (e.g. acne), rhinophyma, or ocular rosacea.

3.40 Any of methods 3.17-3.21, wherein the autoimmune disorder is uticaria.

3.41 Any of methods 3.17-3.21 or 3.40, wherein the autoimmune disorder is chronic spontaneous uticaria.

3.42 Any of methods 3.17-3.21, wherein the autoimmune disorder is Behcet's disease.

3.43 Any of methods 3.17-3.21, wherein the autoimmune disorder is lupus erythematosus.

3.44 Any of methods 3.17-3.21 or 3.43, wherein the autoimmune disorder is systemic lupus erythematosus, discoid lupus erythematosus, or drug-induced lupus.

3.45 Any of methods, 3.17-3.21, wherein the autoimmune disorder is cutaneous graft-versus-host-disease (cGVHD).

3.46 Any of the preceding methods, the subject is a human.

3.47 Any of the preceding methods, wherein the mammalian skin is human skin.

As used herein, “autoimmune disorder” refers to disorders involving the dysregulation of one or more types of T helper cells, e.g., Th17 cells. Autoimmune disorder encompasses various disorders relating to skin inflammation including, for example, psoriasis, atopic dermatitis, and alopecia. Skin inflammation is typically characterized by redness/flushing, pain, pustules, sensation of heat, and/or swelling. The term autoimmune disorder encompasses autoinflammatory disorders, particularly autoinflammatory disorders of the skin.

“Atopic dermatitis” refers to a skin condition involving chronic inflammation, and symptoms of atopic dermatitis include a red, itchy rash. Atopic dermatitis may be present on the skin of any part of the body, but is common on the hands, feet, upper chest, and in the bends of elbows or knees. Additional symptoms of atopic dermatitis may include small raised bumps or thickened, scaly skin.

“Psoriasis” is a chronic skin condition related to an overactive immune response. Psoriasis may be present on may be present on the skin of any part of the body. Symptoms of psoriasis include local inflammation, skin flaking, and thick white or red patches of skin.

“Alopecia” is an autoimmune skin disease, causing hair loss on the scalp, face and sometimes on other areas of the body. In alopecia areata, for example, T cell lymphocytes cluster around affected follicles, causing inflammation and subsequent hair loss.

“Mammal” or “mammalian” includes humans and both domestic animals such as laboratory animals and household pets, (e.g., cats, dogs, swine, cattle, sheep, goats, horses, rabbits), and non-domestic animals such as wildlife and the like.

“Therapeutically effective amount” refers to that amount of a compound of the present disclosure which, when administered to a mammal, preferably a human, is sufficient to effect treatment of the disease or condition of interest in a mammal, preferably a human, having the disease or condition. The amount of a compound of the present disclosure which constitutes a “therapeutically effective amount” will vary depending on the compound, the disease or condition and its severity, the manner of administration, and the age of the mammal to be treated, but can be determined routinely by one of ordinary skill in the art having regard to his own knowledge and to this disclosure. Preferably, for purposes of this disclosure, a “therapeutically effective amount” is that amount of a compound of present disclosure which is sufficient to inhibit inflammation of the skin.

“Treating” or “treatment”, as used herein, covers the treatment of the disease or condition of interest in a mammal, preferably a human, and includes:
(i) preventing the disease or condition from occurring in the mammal;
(ii) inhibiting the disease or condition in the mammal, i.e., arresting its development;
(iii) relieving the disease or condition in the mammal, i.e., causing regression of the disease or condition; or
(iv) relieving the symptoms of the disease or condition in the mammal, i.e., relieving the symptoms without addressing the underlying disease or condition.

As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In the present description, the term “about” means±20% of the indicated range, value, or structure, unless otherwise indicated.

In some embodiments, the RORγt inhibitor (e.g. a RORγt inhibitor according to the present disclosure) is present in the topical composition at a concentration of about 0.001% to about 50% by weight, e.g., a concentration of about 0.01% to about 30% by weight, a concentration of about 0.1% to about 25% by weight, a concentration of about 0.1% to about

20% by weight, or a concentration of about 1% to about 15% by weight, e.g., a concentration of about 1%, 2%, 3%, 4%, 5%, 6%, 7%, 8%, 9%, 10%, 11%, 12%, etc.

In certain embodiments, the pharmaceutical compositions described herein further include a dermatologically acceptable excipient. The dermatologically acceptable excipients may be one or more solvents that solubilize and/or stabilize the active ingredient (e.g., RORγt inhibitors) contained therein. The dermatologically acceptable excipients may also include skin penetration enhancers, preservatives, viscosity enhancers, pH adjusters, film forming agents and the like. Non-limiting examples of the suitable excipients include water, PEG 200, PEG 400, ethanol, glycerol, Transcutol P (diethylene glycol monoethyl ether), propylene glycol, 1,3-dimethyl-2-imidazolidinone (DMI), sodium metabisulfite, butylated hydroxytoluene (BHT), benzyl alcohol, sodium benzoate, isopropyl myristate, diisopropyl adipate, crodamol OHS (ethylhexyl hydroxystearate), mineral oil, Betadex, TWEEN 20, Brij S20 (polyoxyethylene (20) stearyl ether).

More detailed description of certain suitable excipients is described below. As will be appreciated, components of the pharmaceutical formulations described herein can possess multiple functions. For example, a given substance may act as both a viscosity increasing agent and as an emulsifying agent.

The skin (especially stratum corneum) provides a physical barrier to the harmful effects of the external environment. In doing so, it also interferes with the absorption or transdermal delivery of topical therapeutic drugs. Thus, a suitable dermatologically acceptable excipient may include one or more penetration enhancers (or permeation enhancers), which are substances that promote the diffusion of the therapeutic drugs (e.g., the RORγt inhibitors described herein) through the skin barrier. They typically act to reduce the impedance or resistance of the skin to allow improved permeation of the therapeutic drugs. In particular, substances which would perturb the normal structure of the stratum corneum are capable of disrupting the intercellular lipid organization, thus reducing its effectiveness as a barrier. These substances could include any lipid material which would partition into the stratum corneum lipids causing a direct effect or any material which would affect the proteins and cause an indirect perturbation of the lipid structure. Furthermore, solvents, such as ethanol, can remove lipids from the stratum corneum, thus destroying its lipid organization and disrupting its barrier function.

Examples of penetration enhancers or barrier function disrupters include, but are not limited to, alcohol-based enhancers, such as alkanols with one to sixteen carbons, benzyl alcohol, butylene glycol, diethylene glycol, glycofurol, glycerides, glycerin, glycerol, phenethyl alcohol, polypropylene glycol, polyvinyl alcohol, and phenol; amide-based enhancers, such as N-butyl-N-dodecylacetamide, crotamiton, N,N-dimethylformamide, N,N-dimethylacetamide, N-methyl formamide, and urea; amino acids, such as L-α-amino acids and water soluble proteins; azone and azone-like compounds, such as azacycloalkanes; essential oils, such as almond oil, amyl butyrate, apricot kernel oil, avocado oil, camphor, castor oil, 1-carvone, coconut oil, corn oil, cotton seed oil, eugenol, menthol, oil of anise, oil of clove, orange oil, peanut oil, peppermint oil, rose oil, safflower oil, sesame oil, shark liver oil (squalene), soybean oil, sunflower oil, and walnut oil; vitamins and herbs, such as aloe, allantoin, black walnut extract, chamomile extract, panthenol, papain, tocopherol, and vitamin A palmitate; waxes, such as candelilla wax, carnauba wax, ceresin wax, beeswax, lanolin wax, jojoba oil, petrolatum; mixes, such as primary esters of fractionated vegetable oil fatty acids with glycerine or propylene glycol, and interesterified medium chain triglyceride oils; fatty acids and fatty acid esters, such as amyl caproate, butyl acetate, caprylic acid, cetyl ester, diethyl sebacate, dioctyl malate, elaidic acid ethyl caprylate, ethyl glycol palmitostearate, glyceryl beheate, glucose glutamate, isobutyl acetate, laureth-4, lauric acid, malic acid, methyl caprate, mineral oil, myristic acid, oleic acid, palmitic acid, PEG fatty esters, polyoxylene sorbitan monooleate, polypropylene glycols, propylene glycols, saccharose disterate, salicylic acid, sodium citrate, stearic acid, soaps, and caproic-, caprylic-, capric-, and lauric-triglycerides; macrocylics, such as butylated hydroxyanisole, cyclopentadecanolide, cyclodextrins; phospholipid and phosphate enhancers, such as dialkylphosphates, ditetradecyl phosphate, lecithin, 2-pyrrolidone derivatives, such as alkyl pyrrolidone-5-carboxylate esters, pyroglutamic acid esters, N-methyl pyrrolidone, biodegradable soft penetration enhancers, such as dioxane derivatives and dioxolane derivatives; sulphoxide enhancers, such as dimethyl sulphoxide and decylmethyl sulphoxide; acid enhancers, such as alginic acid, sorbic acid, and succinic acid; cyclic amines; imidazolinones; imidazoles; ketones, such as acetone, dimethicone, methyl ethyl ketone, and pentanedione; lanolin derivatives, such as lanolin alcohol, PEG 16 lanolin, and acetylated lanolin; oxazolines; oxazolindinones; proline esters; pyrroles, urethanes; and surfactants, such as nonoxynols, polysorbates, polyoxylene alcohols, polyoxylene fatty acid esters, sodium lauryl sulfate, and sorbitan monostearate.

The topical compositions described herein typically contain one or more carriers, which preferably have a vapor pressure greater than or equal to 23.8 mm Hg at 25□C. Preferred concentration range of a single carrier or the total of a combination of carriers can be from about 0.1 wt. % to about 10 wt. %, more preferably from about 10 wt. % to about 50 wt. %, more specifically from about 50 wt. % to about 95 wt. % of the dermatological composition. Non-limiting examples of the solvent include water (e.g., deionized water) and lower alcohols, including ethanol, 2-propanol and n-propanol.

A dermatological composition of the present disclosure can contain one or more hydrophilic co-solvents, which are miscible with water and/or lower chain alcohols and preferably have a vapor pressure less than water at 25° C. (—23.8 mm Hg). The carrier typically has a vapor pressure greater than or equal to the hydrophilic co-solvent as to concentrate the active ingredient (e.g., a RORγt inhibitor of the present disclosure) on the skin. A hydrophilic co-solvent may be a glycol, specifically propylene glycol. In particular, the propylene glycol can be from the class of polyethylene glycols, specifically polyethylene glycols ranging in molecular weight from 200 to 20000. Preferably, the solvent would be part of a class of glycol ethers. More specifically, a hydrophilic co-solvent of the present disclosure would be diethylene glycol monoethyl ether (transcutol). As used herein, “diethylene glycol monoethyl ether” (“DGME”) or “transcutol” refers to 2-(2-ethoxyethoxy)ethanol {CAS NO 001893} or ethyoxydiglycol. Another preferred co-solvent is 1,3-dimethyl-2-imidazolidinone (DMI).

The topical compositions described herein may also contain one or more “humectant(s)” used to provide a moistening effect. Preferably the humectant remains stable in the composition. Any suitable concentration of a single humectant or a combination of humectants can be employed, provided that the resulting concentration provides the desired moistening effect. Typically, the suitable amount of humectant will depend upon the specific humectant or humectants employed. Preferred concentration range of a single humectant or the total of a combination of humectants can be from about 0.1 wt. % to about 70 wt. %, more preferably from about 5.0 wt. % to about 30 wt. %, more specifically from about 10 wt. % to about 25 wt. % of the dermatological composition. Non-limiting examples for use herein include glycerin, polyhydric alcohols and silicone oils. More preferably, the humectant is glycerin, propylene glycol and/or cyclomethicone. Specifically, the filler would be glycerine and/or cyclomethicone.

In certain embodiments, the pharmaceutical compositions include a viscosity enhancing agent or an emulsifier. Gelling agents are used to increase the viscosity of the final composition. Emulsifiers are substances that stabilize an emulsion. The viscosity increasing agent can also act as an emulsifying agent. Typically, the concentration and combination of viscosity increasing agents will depend on the physical stability of the finished product. Preferred concentration range of a viscosity increasing agent can be from about 0.01 wt. % to about 20 wt. %, more preferably from about 0.1 wt. % to about 10 wt. %, more specifically from about 0.5 wt. % to about 5 wt. % of the dermatological composition. Non-limiting examples of viscosity increasing agents for use herein include classes of celluloses, acrylate polymers and acrylate crosspolymers, such as, hydroxypropyl cellulose, hydroxymethyl cellulose, Pluronic PF127 polymer, carbomer 980, carbomer 1342 and carbomer 940, more preferably hydroxypropyl cellulose, Pluronic PF127 carbomer 980 and carbomer 1342, more specifically hydroxypropyl cellulose (Klucel® EF, GF and/or HF), Pluronic PF127, carbomer 980 and/or carbomer 1342 (Pemulen® TR-1, TR-2 and/or Carbopol® ETD 2020). Examples of emulsifiers for use herein include polysorbates, laureth-4, and potassium cetyl sulfate.

The topical compositions described herein may contain one or more anti-oxidants, radical scavengers, and/or stabilizing agents, preferred concentration range from about 0.001 wt. % to about 0.1 wt. %, more preferably from about 0.1 wt. % to about 5 wt. % of the dermatological composition. Non-limiting examples for use herein include butylatedhydroxytoluene, butylatedhydroxyanisole, ascorbyl palmitate, citric acid, vitamin E, vitamin E acetate, vitamin E-TPGS, ascorbic acid, tocophersolan and propyl gallate. More specifically the anti-oxidant can be ascorbyl palmitate, vitamin E acetate, vitamin E-TPGS, vitamin E or butylatedhydroxy toluene.

The topical compositions described herein may also contain preservatives that exhibit anti-bacterial and/or anti-fungal properties. Preservatives can be present in a gelled dermatological composition of the present disclosure to minimize bacterial and/or fungal over its shelf-life. Preferred concentration range of preservatives in a dermatological composition of the present disclosure can be from about 0.001 wt. % to about 0.01 wt. %, more preferably from about 0.01 wt. % to about 0.5 wt. % of the dermatological composition. Non-limiting examples for use herein include diazolidinyl urea, methylparaben, propylparaben, tetrasodium EDTA, and ethylparaben. More specifically the preservative would be a combination of methylparaben and propylparaben.

The topical compositions described herein may optionally include one or more chelating agents. As used herein, the term “chelating agent” or “chelator” refers to those skin benefit agents capable of removing a metal ion from a system by forming a complex so that the metal ion cannot readily participate in or catalyze chemical reactions. The chelating agents for use herein are preferably formulated at concentrations ranging from about 0.001 wt. % to about 10 wt. %, more preferably from about 0.05 wt. % to about 5.0 wt. % of the dermatological composition. Non-limiting examples for use herein include EDTA, disodium edeate, dipotassium edeate, cyclodextrin, trisodium edetate, tetrasodium edetate, citric acid, sodium citrate, gluconic acid and potassium gluconate. Specifically, the chelating agent can be EDTA, disodium edeate, dipotassium edate, trisodium edetate or potassium gluconate.

The topical compositions described herein may include one or more compatible cosmetically acceptable adjuvants commonly used, such as colorants, fragrances, emollients, and the like, as well as botanicals, such as aloe, chamomile, witch hazel and the like.

Alternatively, other pharmaceutical delivery systems may be employed for the pharmaceutical compositions of the present disclosure. Liposomes and emulsions are well-known examples of delivery vehicles that may be used to deliver active compound(s) or prodrug(s). Certain organic solvents such as dimethylsulfoxide (DMSO) may also be employed.

The topical compositions described herein may be provided in any cosmetically suitable form, preferably as a lotion, a cream, or a ointment, as well as a sprayable liquid form (e.g., a spray that includes the RORγt inhibitor in a base, vehicle or carrier that dries in a cosmetically acceptable way without the greasy appearance that a lotion or ointment would have when applied to the skin).

Any suitable amount of a RORγt inhibitor (e.g., a compound according to the present disclosure) can be employed in such dermatological compositions, provided the amount effectively reduces local inflammation, and remains stable in the composition over a prolonged period of time. Preferably, the stability is over a prolonged period of time, e.g., up to about 3 years, up to 1 year, or up to about 6 months, which is typical in the manufacturing, packaging, shipping and/or storage of dermatologically acceptable compositions. A compound of the present disclosure can be in solution, partially in solution with an undissolved portion or completely undissolved suspension. A compound of the present disclosure can be present in a dermatological composition of the present disclosure in a concentration range from about 0.001 wt. % to about 80 wt. %, from about 0.001 wt. % to about 50 wt. %, from about 0.001 wt. % to about 25 wt. %, or from about 0.001 wt. % to about 6 wt. % of the dermatological composition. In one embodiment, a compound of the present disclosure can be present in a concentration range of from about 0.001 wt. % to about 10 wt. %, from about 0.1 wt. % to about 10 wt. % or from about

1.0 wt. % to about 5.0 wt. % of the dermatological composition.

In treating the autoimmune disorders, e.g., psoriasis, alopecia, or atopic dermatitis, the topical composition comprising a compound of the present disclosure is preferably administered directly to the affected area of the skin (e.g., psoriasis lesion) of the human in need thereof. When such compositions are in use (e.g., when a dermatological composition comprising a compound of the present disclosure) and a dermatologically acceptable excipient is placed upon the skin of the human in need thereof, the RORγt inhibitor of is in continuous contact with the skin of the patient, thereby effecting penetration and treatment.

In topically administering the pharmaceutical compositions of the present disclosure, the skin of the human to be treated can be optionally pre-treated (such as washing the skin with soap and water or cleansing the skin with an alcohol-based cleanser) prior to administration of the dermatological composition of the present disclosure.

The pharmaceutical compositions of the present disclosure may, if desired, be presented in a pack or dispenser device which may contain one or more unit dosage forms containing the active compound(s). The topical composition described herein may also be provided in a patch with the topical composition on the side of the patch that directly contacts the skin. Dermatologically acceptable adhesives may be used to affix the patch to the skin for an extended period of time.

The following Examples may be used by one skilled in the art to determine the effectiveness of the compounds of the present disclosure in treating a human having a dermatological condition characterized by inflammation.

### EXAMPLES

### Example 1-Topical Screening of Compounds for Reduction of Inflammation

A skin Resident Immune Cell Assay (sRICA) was used to test compounds for reduction of IL-17A protein upregulation in the Th17 sRICA model. In this model, human surgical skin waste was cultured in a transwell system, with the dermis in contact with cell culture media and the stratum corneum exposed to air. To perform the assay, each human skin sample was defatted and dermatomed to 750μm. Next, 8 mm punch biopsies were obtained and placed in a membrane transwell. The transwells were inserted into culture wells with complete media, and a cocktail of cytokines and antibodies were added to promote skin resident immune cell polarization.

RORγt inhibitors were prepared as a 100 uM solution in 1% DMSO/OD. 100 of the prepared topical solution was applied to sRICA wells prepared for topical drug evaluation. Drugs were allowed to penetrate the skin overnight, for up to 24 hours prior to Th17 activation. Media was collected 24 hours after Th17 activation for IL17A protein assessment. IL17A protein was evaluated using the Quanterix IL17A detection kit on the SiMoA platform (via CRO Translational Biosciences). There was significant upregulation of IL17A with Th17 activation. Each study was normalized to the % max of IL17A production (i.e. the average IL17A concentration of Th17 activated skin alone) was set to 100%, all other values (untreated no activation and treated samples) were obtained as a percentage of this.

Results are summarized below in Table 1.

TABLE 1
Results for Select Compounds in Comparison with Controls

| | | Activity in |
|---|---|---|
| | | sRICA |
| | | (% inhibition |
| Compound Structure | Compound name | of IL17A) |
| 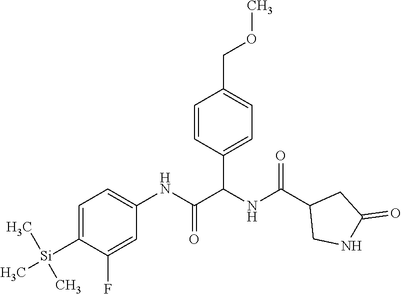 [SMILES: COCc1ccc(C(NC(=O)C2CNC(=O)C2)C(=O)Nc2ccc([Si](C)(C)C)c(F)c2)cc1] | N-(2-((3-fluoro-4- (trimethylsilyl)phenyl)amino)-1-(4- (methoxymethyl)phenyl)-2-oxoethyl)-5- oxopyrrolidine-3-carboxamide | 85.24 |
| 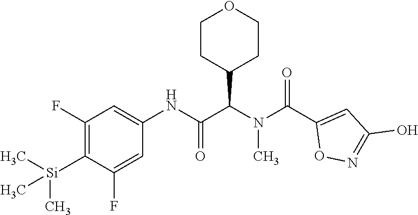 [SMILES: CN(C(=O)c1cc(O)no1)[C@@H](C(=O)Nc1cc(F)c([Si](C)(C)C)c(F)c1)C1CCOCC1] | N-(2-((3,5-difluoro-4- (trimethylsilyl)phenyl)amino)-2-oxo-1- (tetrahydro-2H-pyran-4-yl)ethyl)-3- hydroxy-N-methyl-1,2-oxazole-5- carboxamide | 82.49 |
| 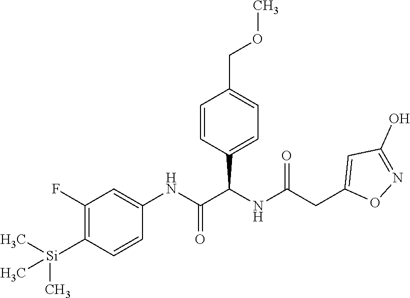 [SMILES: COCc1ccc([C@@H](NC(=O)Cc2cc(O)no2)C(=O)Nc2ccc([Si](C)(C)C)c(F)c2)cc1] | N-(1-(4-methoxyphenyl)-2-oxo-2-((4- (trimethylsilyl)phenyl)amino)ethyl)-N- methyl-1,2-oxazole-5-carboxamide | 81.62 |
| 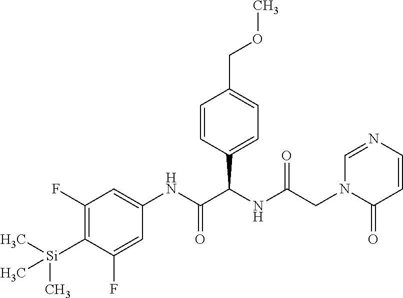 [SMILES: COCc1ccc([C@@H](NC(=O)Cn2cnccc2=O)C(=O)Nc2cc(F)c([Si](C)(C)C)c(F)c2)cc1] | (2R)-N-(3,5-difluoro-4- (trimethylsilyl)phenyl)-2-(4- (methoxymethyl)phenyl)-2-(((6- oxopyrimidin-1(6H)- yl)acetyl)amino)acetamide | 80.01 |
| 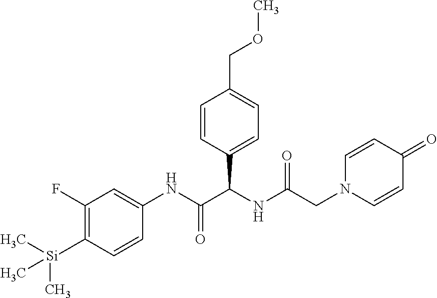 [SMILES: COCc1ccc([C@@H](NC(=O)Cn2ccc(=O)cc2)C(=O)Nc2ccc([Si](C)(C)C)c(F)c2)cc1] | N-(1-4-methoxyphenyl)-2-oxo-2-((4- (trimethylsilyl)phenyl)amino)ethyl)-N- methyltetrahydrothiophene-3- carboxamide-1,1-dioxide | 78.43 |
| 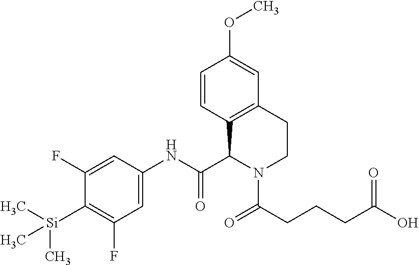 [SMILES: COc1ccc2c(c1)CCN(C(=O)CCCC(=O)O)[C@H]2C(=O)Nc1cc(F)c([Si](C)(C)C)c(F)c1] | 5-((1R)-1-((3,5-difluoro-4- (trimethylsilyl)phenyl)carbamoyl)-6- methoxy-3,4-dihydroisoquinolin-2(1H)- yl)-5-oxopentanoic acid | 76.57 |
| 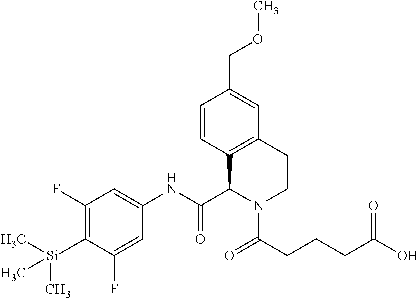 [SMILES: COCc1ccc2c(c1)CCN(C(=O)CCCC(=O)O)[C@H]2C(=O)Nc1cc(F)c([Si](C)(C)C)c(F)c1] | 5-((1R)-1-((3,5-difluoro-4- (trimethylsilyl)phenyl)carbamoyl)-6- methoxymethyl-3,4-dihydroisoquinolin- 2(1H)-yl)-5-oxopentanoic acid | 77.39 |
| 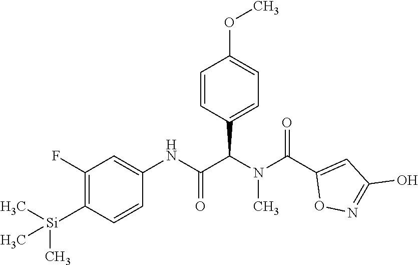 [SMILES: COc1ccc([C@H](C(=O)Nc2ccc([Si](C)(C)C)c(F)c2)N(C)C(=O)c2cc(O)no2)cc1] | N-((1R)-2-((3-fluoro-4- (trimethylsilyl)phenyl)amino-1-(4- methoxyphenyl)-2-oxoethyl)-3-hydroxy- N-methyl-1,2-oxazole-5-carboxamide | 76.87 |
| 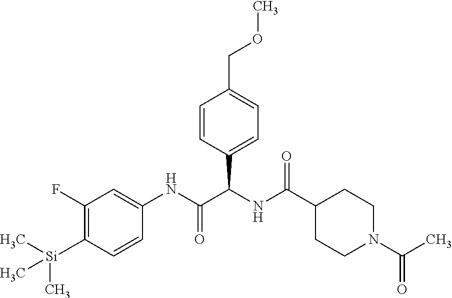 [SMILES: COCc1ccc([C@@H](NC(=O)C2CCN(C(C)=O)CC2)C(=O)Nc2ccc([Si](C)(C)C)c(F)c2)cc1] | 1-acetyl-N-((1R)-2-((3-fluoro-4- (trimethylsilyl)phenyl)amino)-1-(4- (methoxymethyl)phenyl)-2- oxoethyl)piperidine-4-carboxamide | 77.49 |
| 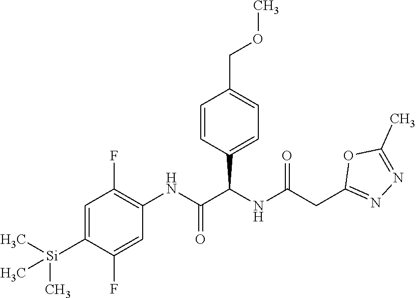 [SMILES: COCc1ccc([C@@H](NC(=O)Cc2nnc(C)o2)C(=O)Nc2cc(F)c([Si](C)(C)C)cc2F)cc1] | (2R)-N-(2,5-difluoro-4- (trimethylsilyl)phenyl)-2-(4- (methoxymethyl)phenyl)-2-(((5-methyl- 1,3,4-oxadiazol-2- yl)acetyl)amino)acetamide | 75.72 |
| 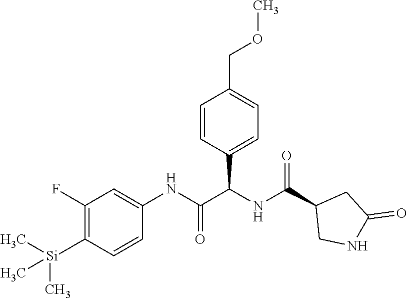 [SMILES: COCc1ccc([C@@H](NC(=O)[C@@H]2CNC(=O)C2)C(=O)Nc2ccc([Si](C)(C)C)c(F)c2)cc1] | (3R)-N-((1R)-2-((3-fluoro-4- (trimethylsilyl)phenyl)amino)-1-(4- (methoxymethyl)phenyl)-2-oxoethyl)-5- oxopyrrolidine-3-carboxamide | 75.70 |
| 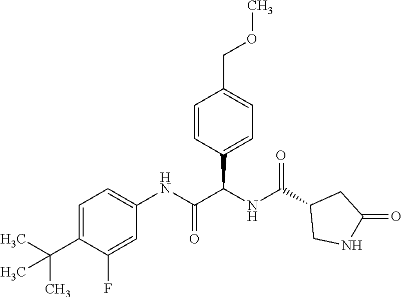 [SMILES: COCc1ccc([C@@H](NC(=O)[C@H]2CNC(=O)C2)C(=O)Nc2ccc(C(C)(C)C)c(F)c2)cc1] | (3R)-N-((1R)-2-((4-tert-butyl-3- fluorophenyl)amino)-1-(4- (methoxymethyl)phenyl)-2-oxoethyl)-5- oxopyrrolidine-3-carboxamide | 74.06 |
| 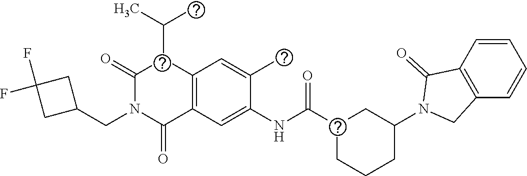 [SMILES: Cc1cc2c(cc1NC(=O)C1CCCC(N3Cc4ccccc4C3=O)C1)C(=O)N(CC1CC(F)(F)C1)C(=O)C2C(C)C] | (3R)-N-(3-((3,3-difluorocyclobutyl)- methyl)-7-fluoro-1-isopropyl-2,4-dioxo- 1,2,3,4-tetrahydro-quinazolin-6-yl)-3-(1- oxo-1,3-dihydro-2H-isoindol-2-yl)- piperidine-1-carboxamide | 73.12 |
| 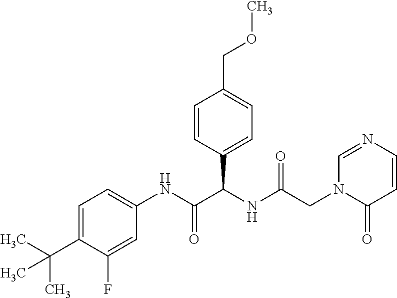 [SMILES: COCc1ccc([C@@H](NC(=O)Cn2cnccc2=O)C(=O)Nc2ccc(C(C)(C)C)c(F)c2)cc1] | (2R)-N-(4-(tert-butyl-3-fluorophenyl)-2- (4-(methoxymethyl)phenyl)-2-(((6- oxopyrimidin-1(6H)- yl)acetyl)amino)acetamide | 72.12 |
| 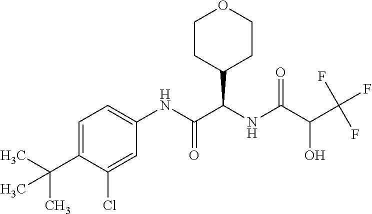 [SMILES: CC(C)(C)c1ccc(NC(=O)[C@H](NC(=O)C(O)C(F)(F)F)C2CCOCC2)cc1Cl] | N-((1R)-2-((4-tert-butyl-3- chlorophenyl)amino)-2-oxo-1- (tetrahydro-2H-pyran-4-yl)ethyl)-3,3,3- trifluoro-2-hydroxypropanamide | 71.26 |
| 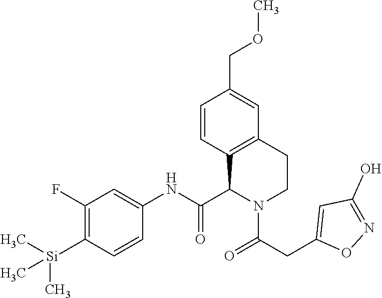 [SMILES: COCc1ccc2c(c1)CCN(C(=O)Cc1cc(O)no1)[C@H]2C(=O)Nc1ccc([Si](C)(C)C)c(F)c1] | (1R)-N-(3-fluoro-4- (trimethylsilyl)phenyl)-2-((3-hydroxy- 1,2-oxazol-5-yl)acetyl)-6- (methoxymethyl)-1,2,3,4- tetrahydroisoquinoline-1-carboxamide | 69.90 |
| 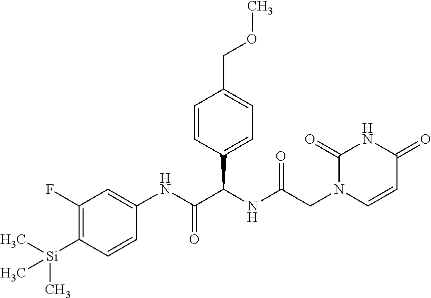 [SMILES: COCc1ccc([C@@H](NC(=O)Cn2ccc(=O)[nH]c2=O)C(=O)Nc2ccc([Si](C)(C)C)c(F)c2)cc1] | (2R)-2-(((2,4-dioxo-3,4- dihydropyrimidin-1(2H)- yl)acetyl)amino)-N-(3-fluoro-4- (trimethylsilyl)phenyl)-2-(4- (methoxymethyl)phenyl)acetamide | 68.63 |
| 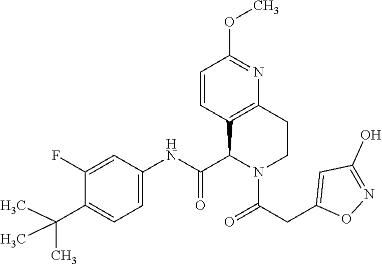 [SMILES: COc1ccc2c(n1)CCN(C(=O)Cc1cc(O)no1)[C@H]2C(=O)Nc1ccc(C(C)(C)C)c(F)c1] | (5R)-N-(4-tert-butyl-3-fluorophenyl)-6- ((3-hydroxy-1,2-oxazol-5-yl)acetyl)-2- methoxy-5,6,7,8-tetrahydro-1,6- naphthyridine-5-carboxamide | 69.49 |
| 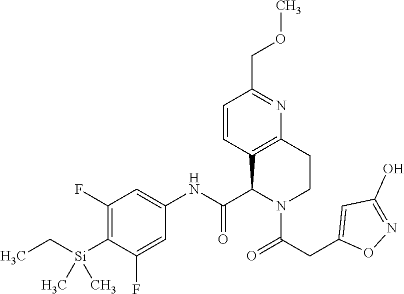 [SMILES: CC[Si](C)(C)c1c(F)cc(NC(=O)[C@H]2c3ccc(COC)nc3CCN2C(=O)Cc2cc(O)no2)cc1F] | (5R)-N-(-4-(ethyl(trimethylsilyl)-3,5- difluorophenyl)-6-((3-hydroxy-1,2- oxazol-5-yl)acetyl)-2-methoxymethyl- 5,6,7,8-tetrahydro-1,6-naphthyridine-5- carboxamide | 68.88 |
| 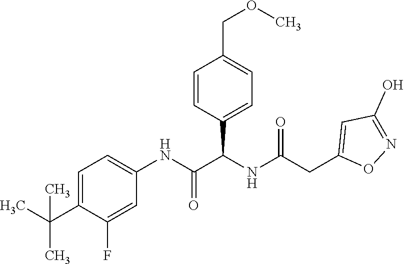 [SMILES: COCc1ccc([C@@H](NC(=O)Cc2cc(O)no2)C(=O)Nc2ccc(C(C)(C)C)c(F)c2)cc1] | (2R)-N-(4-tert-butyl-3-fluorophenyl)-2- (((3-hydroxy-1,2-oxazol-5- yl)acetyl)amino)-2-(4- (methoxymethyl)phenyl)acetamide | 68.13 |
| 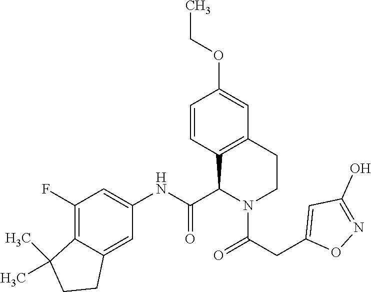 [SMILES: CCOc1ccc2c(c1)CCN(C(=O)Cc1cc(O)no1)[C@H]2C(=O)Nc1cc(F)c2c(c1)CCC2(C)C] | (1R)-6-ethoxy-N-(7-fluoro-1,1-dimethyl- 2,3-dihydro-1H-inden-5-yl)-2-((3- hydroxy-1,2-oxazol-5-yl)acetyl)-1,2,3,4- tetrahydroisoquinoline-1-carboxamide | 67.87 |
| 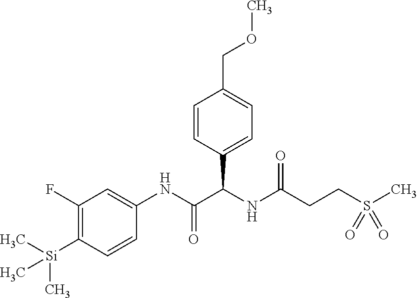 | N-((1R)-2-((3-fluoro-4- (trimethylsilyl)phenyl)amino)-1-(4- (methoxymethyl)phenyl)-2-oxoethyl)-3- (methylsulfonyl)propanamide | 67.36 |
| 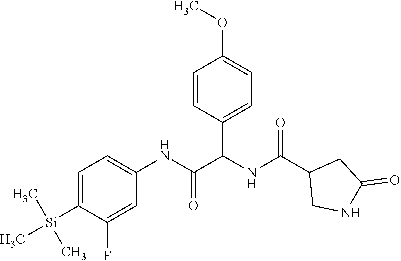 [SMILES: COc1ccc(C(NC(=O)C2CNC(=O)C2)C(=O)Nc2ccc([Si](C)(C)C)c(F)c2)cc1] | N-(2-((3-fluoro-4- (trimethylsilyl)phenyl)amino)-1-(4- methoxyphenyl)-2-oxoethyl)-5- oxopyrrolidine-3-carboxamide | 65.76 |
| 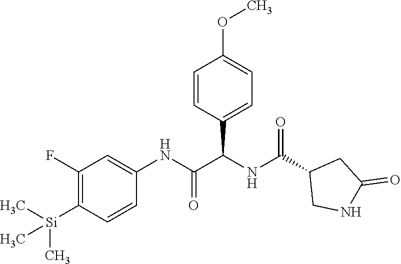 [SMILES: COc1ccc([C@@H](NC(=O)[C@H]2CNC(=O)C2)C(=O)Nc2ccc([Si](C)(C)C)c(F)c2)cc1] | (3R)-N-((1R)-2-((3-fluoro-4- (trimethylsilyl)phenyl)amino)-1-(4- (methoxy)phenyl)-2-oxoethyl)-5- oxopyrrolidine-3-carboxamide | 65.16 |
| 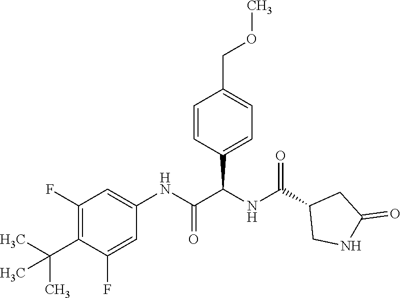 [SMILES: COCc1ccc([C@@H](NC(=O)[C@H]2CNC(=O)C2)C(=O)Nc2cc(F)c(C(C)(C)C)c(F)c2)cc1] | (3R)-N-((1R)-2-((4-tert-butyl-3,5- difluorophenyl)amino)-1-(4- (methoxymethyl)phenyl)-2-oxoethyl)-5- oxopyrrolidine-3-carboxamide | 63.07 |
| 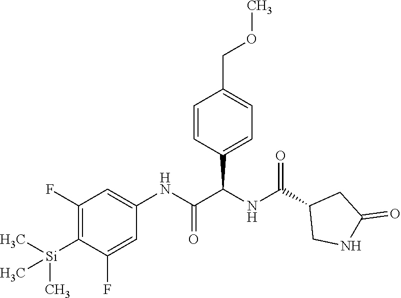 [SMILES: COCc1ccc([C@@H](NC(=O)[C@H]2CNC(=O)C2)C(=O)Nc2cc(F)c([Si](C)(C)C)c(F)c2)cc1] | (3R)-N-((1R)-2-((3,5-difluoro-4- (trimethylsilyl)phenyl)amino)-1-(4- (methoxymethyl)phenyl)-2-oxoethyl)-5- oxopyrrolidine-3-carboxamide | 63.00 |
| 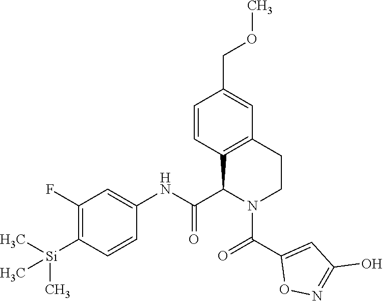 [SMILES: COCc1ccc2c(c1)CCN(C(=O)c1cc(O)no1)[C@H]2C(=O)Nc1ccc([Si](C)(C)C)c(F)c1] | (1R)-N-(3-fluoro-4- (trimethylsilyl)phenyl)-2-((3-hydroxy- 1,2-oxazol-5-yl)carbonyl)-6- methoxymethyl-1,2,3,4- tetrahydroisoquinoline-1-carboxamide | 62.25 |
| 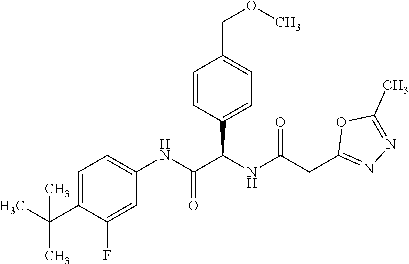 [SMILES: COCc1ccc([C@@H](NC(=O)Cc2nnc(C)o2)C(=O)Nc2ccc(C(C)(C)C)c(F)c2)cc1] | (2R)-N-(4-tert-butyl-3-fluorophenyl)-2- (4-(methoxymethyl)phenyl)-2-(((5- methyl-1,3,4-oxadiazol-2- yl)acetyl)amino)acetamide | 62.05 |
| 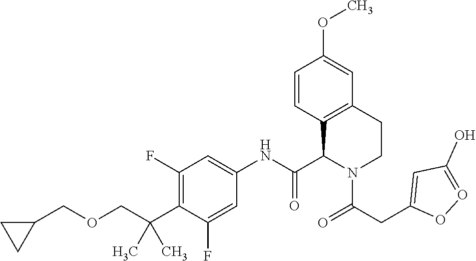 | (1R)-N-(4-(1-(cyclopropyl-methoxy)-2- methylpropan-2-yl)-3,5-difluorophenyl)- 2-((3-hydroxy-1,2-oxazol-5-yl)acetyl)-6- methoxy-1,2,3,4-tetrahydro-isoquinoline- 1-carboxamide | 61.74 |
| 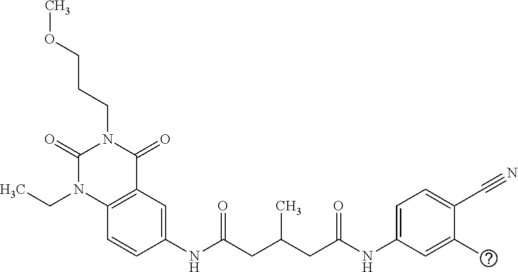 [SMILES: CCn1c(=O)n(CCCOC)c(=O)c2cc(NC(=O)CC(C)CC(=O)Nc3ccc(C#N)c(C)c3)ccc21] | N-(3-chloro-4-cyanophenyl)-N′-(1-ethyl- 3-(3-methoxypropyl)-2,4-dioxo-1,2,3,4- tetrahydroquinazolin-6-yl)-3- methylpentanediamide | 60.80 |
| 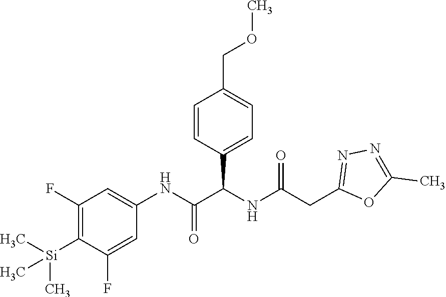 | (2R)-N-(3,5-difluoro-4- (trimethylsilyl)phenyl)-2-(4- (methoxymethyl)phenyl)-2-(((5-methyl- 1,3,4-oxadiazol-2- yl)acetyl)amino)acetamide | 60.79 |
| 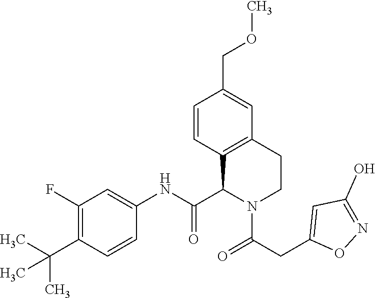 [SMILES: COCc1ccc2c(c1)CCN(C(=O)Cc1cc(O)no1)[C@H]2C(=O)Nc1ccc(C(C)(C)C)c(F)c1] | (1R)-N-(4-tert-butyl-3-fluorophenyl)-2- ((3-hydroxy-1,2-oxazol-5-yl)acetyl)-6- methoxymethyl-1,2,3,4- tetrahydroisoquinoline-1-carboxamide | 60.77 |
| 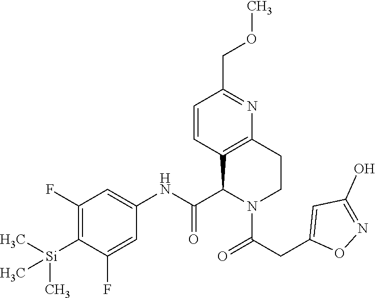 [SMILES: COCc1ccc2c(n1)CCN(C(=O)Cc1cc(O)no1)[C@H]2C(=O)Nc1cc(F)c([Si](C)(C)C)c(F)c1] | (5R)-N-(3,5-difluoro-4- (trimethylsilyl)phenyl)-6-((3-hydroxy- 1,2-oxazol-5-yl)acetyl)-2- (methoxymethyl)-5,6,7,8-tetrahydro-1,6- naphthyridine-5-carboxamide | 60.13 |
| 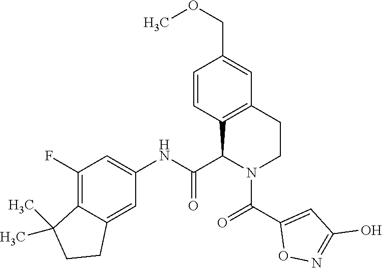 [SMILES: COCc1ccc2c(c1)CCN(C(=O)c1cc(O)no1)[C@H]2C(=O)Nc1cc(F)c2c(c1)CCC2(C)C] | (1R)-N-(7-fluoro-1,1-dimethyl-2,3- dihydro-1H-inden-5-yl)-2-((3-hydroxy- 1,2-oxazol-5-yl)carbonyl)-6- (methoxymethyl)-1,2,3,4- tetrahydroisoquinoline-1-carboxamide | 59.11 |
| 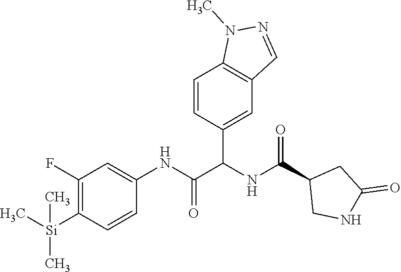 [SMILES: Cn1ncc2cc(C(NC(=O)[C@@H]3CNC(=O)C3)C(=O)Nc3ccc([Si](C)(C)C)c(F)c3)ccc21] | (3S)-N-(2-((3-fluoro-4- (trimethylsilyl)phenyl)amino)-1-(1- methyl-1H-indazol-5-yl)-2-oxoethyl)-5- oxopyrrolidine-3-carboxamide | 58.27 |
| 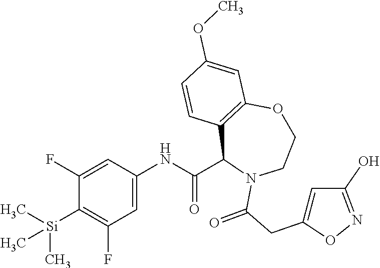 [SMILES: COc1ccc2c(c1)OCCN(C(=O)Cc1cc(O)no1)[C@H]2C(=O)Nc1cc(F)c([Si](C)(C)C)c(F)c1] | (5R)-N-(3,5-difluoro-4- (trimethylsilyl)phenyl)-4-((3-hydroxy- 1,2-oxazol-5-yl)acetyl)-8-methoxy- 2,3,4,5-tetrahydro-1,4-benzoxazepine-5- carboxamide | 58.07 |
| 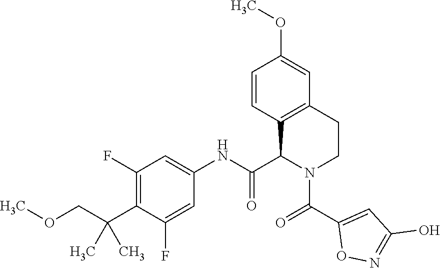 [SMILES: COCC(C)(C)c1c(F)cc(NC(=O)[C@H]2c3ccc(OC)cc3CCN2C(=O)c2cc(O)no2)cc1F] | (1R)-N-(3,5-difluoro-4-(1-methoxy-2- methylpropan-2-yl)phenyl)-2-((3- hydroxy-1,2-oxazol-5-yl)carbonyl)-6- methoxy-1,2,3,4-tetrahydroisoquinoline- 1-carboxamide | 56.16 |
| 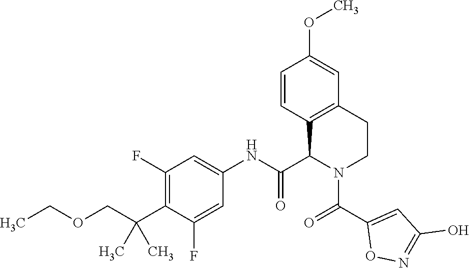 [SMILES: CCOCC(C)(C)c1c(F)cc(NC(=O)[C@H]2c3ccc(OC)cc3CCN2C(=O)c2cc(O)no2)cc1F] | (1R)-N-(4-(1-ethoxy-2-methylpropan-2- yl)-3,5-difluorophenyl)-2-((3-hydroxy- 1,2-oxazol-5-yl)carbonyl)-6-methoxy- 1,2,3,4-tetrahydro-isoquinoline-1- carboxamide | 56.08 |
| 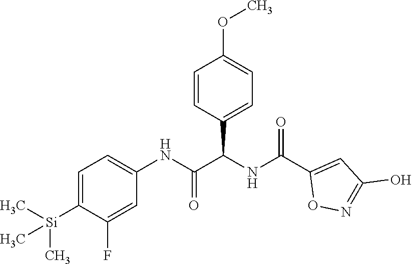 [SMILES: COc1ccc([C@@H](NC(=O)c2cc(O)no2)C(=O)Nc2ccc([Si](C)(C)C)c(F)c2)cc1] | N-((1R)-2-((3-fluoro-4- (trimethylsilyl)phenyl)amino)-1-(4- (methoxyphenyl)-2-oxoethyl)-3-hydroxy- 1,2-oxazole-5-carboxamide | 55.31 |
| 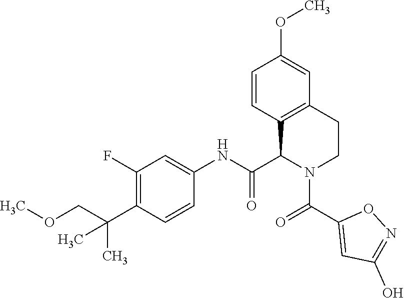 [SMILES: COCC(C)(C)c1ccc(NC(=O)[C@H]2c3ccc(OC)cc3CCN2C(=O)c2cc(O)no2)cc1F] | (1R)-N-(3-fluoro-4-(1-methoxy-2- methylpropan-2-yl)phenyl)-2-((3- hydroxy-1,2-oxazol-5-yl)carbonyl)-6- methoxy-1,2,3,4-tetrahydroisoquinoline- 1-carboxamide | 55.05 |
| 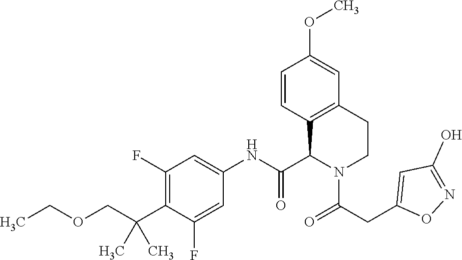 [SMILES: CCOCC(C)(C)c1c(F)cc(NC(=O)[C@H]2c3ccc(OC)cc3CCN2C(=O)Cc2cc(O)no2)cc1F] | (1R)-N-(4-(1-ethoxy-2-methylpropan-2- yl)-3,5-difluorophenyl)-2-((3-hydroxy- 1,2-oxazol-5-yl)acetyl)-6-methoxy- 1,2,3,4-tetrahydro-isoquinoline-1- carboxamide | 54.92 |
| 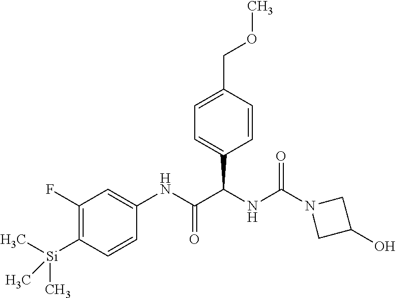 [SMILES: COCc1ccc([C@@H](NC(=O)N2CC(O)C2)C(=O)Nc2ccc([Si](C)(C)C)c(F)c2)cc1] | N-((1R)-2-((3-fluoro-4- (trimethylsilyl)phenyl)amino)-1-(4- (methoxymethyl)phenyl)-2-oxoethyl)-3- hydroxyazetidine-1-carboxamide | 54.62 |
| 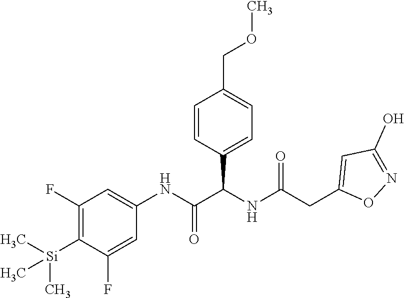 [SMILES: COCc1ccc([C@@H](NC(=O)Cc2cc(O)no2)C(=O)Nc2cc(F)c([Si](C)(C)C)c(F)c2)cc1] | (2R)-N-(3,5-difluoro-4- (trimethylsilyl)phenyl)-2-(((3-hydroxy- 1,2-oxazol-5-yl)acetyl)amino)-2-(4- (methoxymethyl)phenyl)acetamide | 53.91 |
| 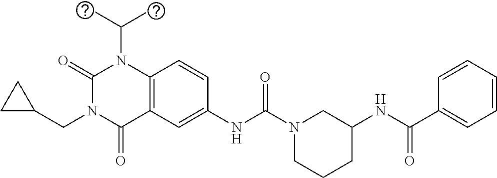 [SMILES: CC(C)n1c(=O)n(CC2CC2)c(=O)c2cc(NC(=O)N3CCCC(NC(=O)c4ccccc4)C3)ccc21] | (3R)-3-(benzoylamino)-N-(3- (cyclopropylmethyl)-1-isopropyl-2,4- dioxo-1,2,3,4-tetrahydroquinazolin-6- yl)piperidine-1-carboxamide | 53.19 |
| 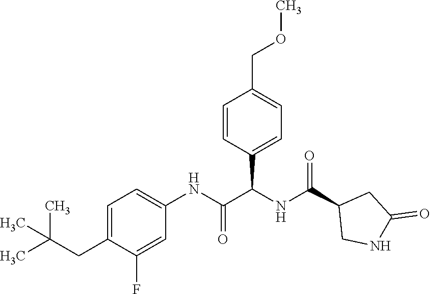 [SMILES: COCc1ccc([C@@H](NC(=O)[C@@H]2CNC(=O)C2)C(=O)Nc2ccc(CC(C)(C)C)c(F)c2)cc1] | (3S)-N-((1R)-2-((4-(2,2-dimethylpropyl)- 3-fluorophenyl)amino)-1-(4- (methoxymethyl)phenyl)-2-oxoethyl)-5- oxopyrrolidine-3-carboxamide | 52.87 |
| 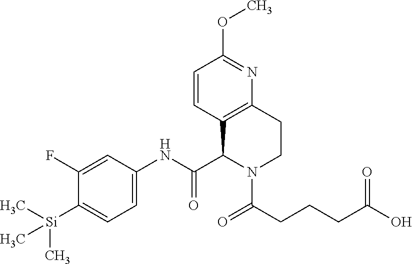 [SMILES: COc1ccc2c(n1)CCN(C(=O)CCCC(=O)O)[C@H]2C(=O)Nc1ccc([Si](C)(C)C)c(F)c1] | 5-((5R)-5-((3-fluoro-4- (trimethylsilyl)phenyl)carbamoyl)-2- methoxy-7,8-dihydro-1,6-naphthyridin- 6(5H)-yl)-5-oxopentanoic acid | 51.48 |
| 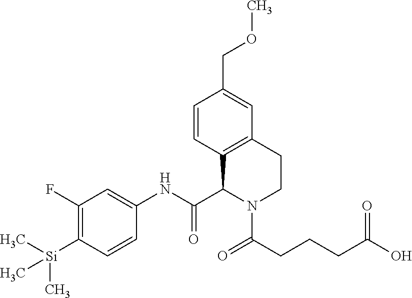 [SMILES: COCc1ccc2c(c1)CCN(C(=O)CCCC(=O)O)[C@H]2C(=O)Nc1ccc([Si](C)(C)C)c(F)c1] | 5-((1R)-1-((3-fluoro-4- (trimethylsilyl)phenyl)carbamoyl)-6- (methoxymethyl)-3,4-dihydroisoquinolin- 2(1H)-yl)-5-oxopentanoic acid | 51.14 |
| 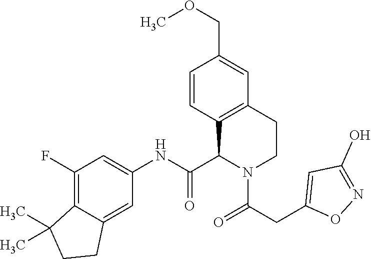 [SMILES: COCc1ccc2c(c1)CCN(C(=O)Cc1cc(O)no1)[C@H]2C(=O)Nc1cc(F)c2c(c1)CCC2(C)C] | (1R)-N-(7-fluoro-1,1-dimethyl-2,3- dihydro-1H-inden-5-yl)-2-((3-hydroxy- 1,2-oxazol-5-yl)acetyl)-6- (methoxymethyl)-1,2,3,4- tetrahydroisoquinoline-1-carboxamide | 50.53 |
| 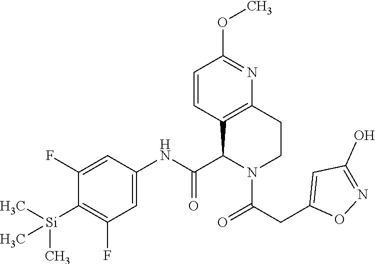 [SMILES: COc1ccc2c(n1)CCN(C(=O)Cc1cc(O)no1)[C@H]2C(=O)Nc1cc(F)c([Si](C)(C)C)c(F)c1] | (5R)-N-(3,5-difluoro-4- (trimethylsilyl)phenyl)-6-((3-hydroxy- 1,2-oxazol-5-yl)acetyl)-2-methoxy- 5,6,7,8-tetrahydro-1,6-naphthyridine-5- carboxamide | 49.41 |
| 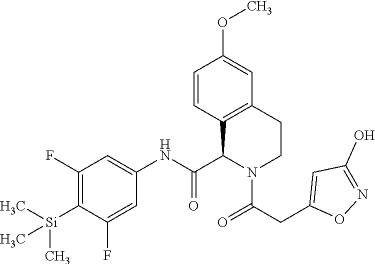 [SMILES: COc1ccc2c(c1)CCN(C(=O)Cc1cc(O)no1)[C@H]2C(=O)Nc1cc(F)c([Si](C)(C)C)c(F)c1] | (1R)-N-(3,5-difluoro-4- (trimethylsilyl)phenyl)-2-((3-hydroxy- 1,2-oxazol-5-yl)acetyl)-6-methoxy- 1,2,3,4-tetrahydroisoquinoline-1- carboxamide | 49.20 |
| 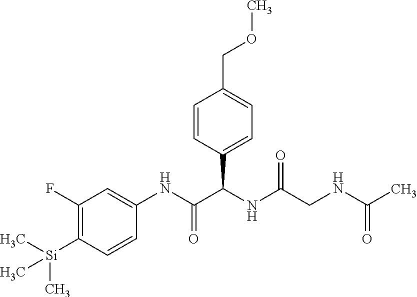 [SMILES: COCc1ccc([C@@H](NC(=O)CNC(C)=O)C(=O)Nc2ccc([Si](C)(C)C)c(F)c2)cc1] | (R)-2-(2-acetamidoacetamido)-N-(3- fluoro-4-(trimethylsilyl)phenyl)-2-(4- (methoxymethyl)phenyl)acetamide) | 48.76 |
| 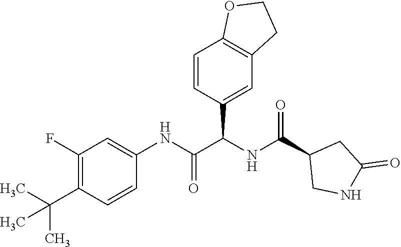 [SMILES: CC(C)(C)c1ccc(NC(=O)[C@H](NC(=O)[C@@H]2CNC(=O)C2)c2ccc3c(c2)CCO3)cc1F] | (3S)-N-(2-((4-tert-butyl-3- fluorophenyl)amino)-1-(2,3-dihydro-1- benzofuran-5-yl)-2-oxoethyl)-5- oxopyrrolidine-3-carboxamide | 48.71 |
| 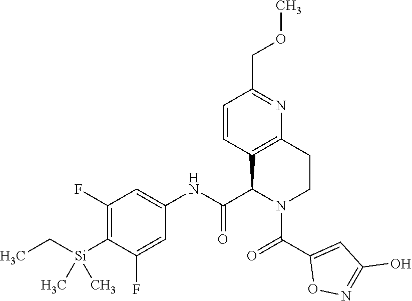 [SMILES: CC[Si](C)(C)c1c(F)cc(NC(=O)[C@H]2c3ccc(COC)nc3CCN2C(=O)c2cc(O)no2)cc1F] | (5R)-N-(4-(ethyl(dimethyl)silyl)-3,5- difluorophenyl)-6-((3-hydroxy-1,2- oxazol-5-yl)carbonyl)-2- (methoxymethyl)-5,6,7,8-tetrahydro-1,6- naphthyridine-5-carboxamide | 48.49 |
| 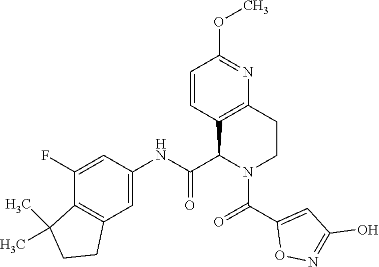 [SMILES: COc1ccc2c(n1)CCN(C(=O)c1cc(O)no1)[C@H]2C(=O)Nc1cc(F)c2c(c1)CCC2(C)C] | (5R)-N-(7-fluoro-1,1-dimethyl-2,3- dihydro-1H-inden-5-yl)-6-((3-hydroxy- 1,2-oxazol-5-yl)carbonyl)-2-methoxy- 5,6,7,8-tetrahydro-1,6-naphthyridine-5- carboxamide | 46.89 |
| 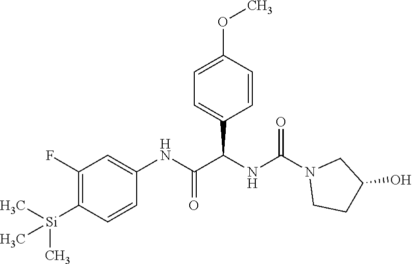 [SMILES: COc1ccc([C@@H](NC(=O)N2CC[C@@H](O)C2)C(=O)Nc2ccc([Si](C)(C)C)c(F)c2)cc1] | (3R)-N-((1R)-2-((3-fluoro-4- (trimethylsilyl)phenyl)amino)-1-(4- methoxyphenyl)-2-oxoethyl)-3- hydroxypyrrolidine-1-carboxamide | 46.88 |
| 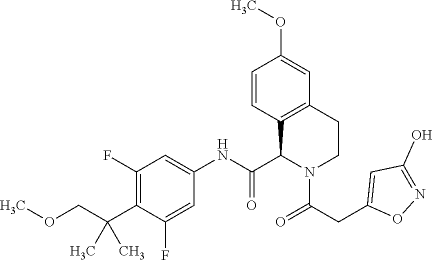 [SMILES: COCC(C)(C)c1c(F)cc(NC(=O)[C@H]2c3ccc(OC)cc3CCN2C(=O)Cc2cc(O)no2)cc1F] | (1R)-N-(3,5-difluoro-4-(1-methoxy-2- methylpropan-2-yl)phenyl)-6-methoxy-2- ((3-hydroxy-1,2-oxazol-5-yl)acetyl)- 1,2,3,4-tetrahydro-isoquinoline-1- carboxamide | 46.29 |
| 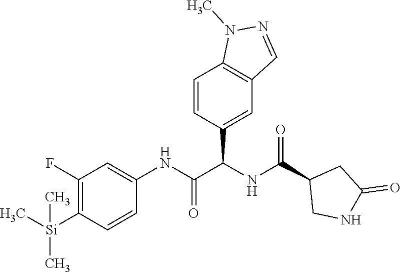 [SMILES: Cn1ncc2cc([C@@H](NC(=O)[C@@H]3CNC(=O)C3)C(=O)Nc3ccc([Si](C)(C)C)c(F)c3)ccc21] | (3S)-N-((1R)-2-((3-fluoro-4- (trimethylsilyl)phenyl)amino)-1-(1- methyl-1H-indazol-5-yl)-2-oxoethyl)-5- oxopyrrolidine-3-carboxamide | 45.71 |
| 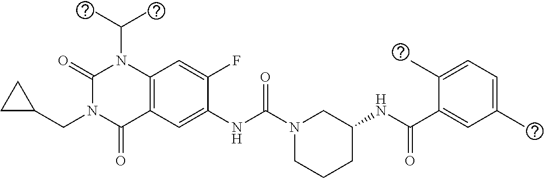 [SMILES: Cc1ccc(C)c(C(=O)N[C@@H]2CCCN(C(=O)Nc3cc4c(=O)n(CC5CC5)c(=O)n(C(C)C)c4cc3F)C2)c1] | (3R)-N-(3-(cyclopropylmethyl)-7-fluoro- 1-isopropyl-2,4-dioxo-1,2,3,4- tetrahydroquinazolin-6-yl)-3-((2,5- difluorobenzoyl)amino)-piperidine-1- carboxamide | 44.19 |
| 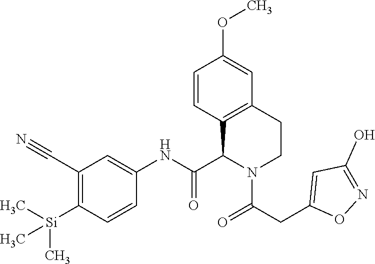 [SMILES: COc1ccc2c(c1)CCN(C(=O)Cc1cc(O)no1)[C@H]2C(=O)Nc1ccc([Si](C)(C)C)c(C#N)c1] | (1R)-N-(3-cyano-4- (trimethylsilyl)phenyl)-2-((3-hydroxy- 1,2-oxazol-5-yl)acetyl)-6-methoxy- 1,2,3,4-tetrahydroisoquinoline-1- carboxamide | 42.78 |
| 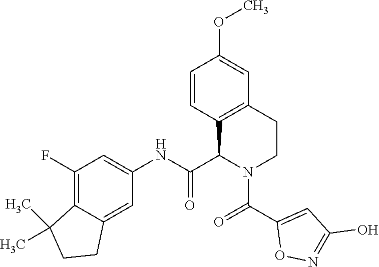 [SMILES: COc1ccc2c(c1)CCN(C(=O)c1cc(O)no1)[C@H]2C(=O)Nc1cc(F)c2c(c1)CCC2(C)C] | N-(7-fluoro-1,1-dimethyl-2,3-dihydro- 1H-inden-5-yl)-2-((3-hydroxy-1,2-oxazol- 5-yl)carbonyl)-6-methoxy-1,2,3,4- tetrahydroisoquinoline-1-carboxamide | 42.30 |
| 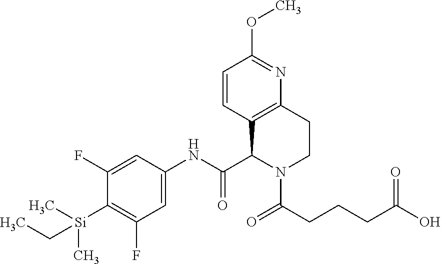 [SMILES: CC[Si](C)(C)c1c(F)cc(NC(=O)[C@H]2c3ccc(OC)nc3CCN2C(=O)CCCC(=O)O)cc1F] | 5-((5R)-5-((4-(ethyl(dimethyl)silyl)-3,5- difluorophenyl)carbamoyl)-2-methoxy- 7,8-dihydro-1,6-naphthyridin-6(5H)-yl)- 5-oxopentanoic acid | 41.92 |
| 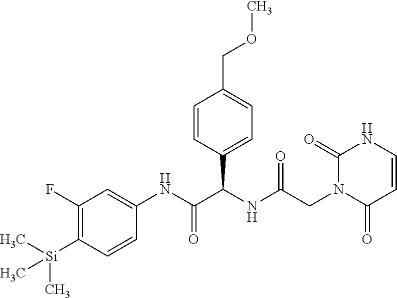 [SMILES: COCc1ccc([C@@H](NC(=O)Cn2c(=O)cc[nH]c2=O)C(=O)Nc2ccc([Si](C)(C)C)c(F)c2)cc1] | (2R)-2-((2,6-dioxo-3,6- dihydropyrimidin-1(2H)- yl)acetyl)amino)-N-(3-fluoro-4- (trimethylsilyl)phenyl)-2-(4- (methoxymethyl)phenyl)acetamide | 40.73 |
| 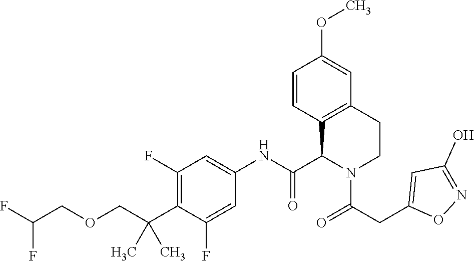 [SMILES: COc1ccc2c(c1)CCN(C(=O)Cc1cc(O)no1)[C@H]2C(=O)Nc1cc(F)c(C(C)(C)COCC(F)F)c(F)c1] | (1R)-N-(4-(1-(2,2-difluoroethoxy)-2- methylpropan-2-yl)-3,5-difluorophenyl)- 2-((3-hydroxy-1,2-oxazol-5-yl)acetyl)-6- methoxy-1,2,3,4-tetrahydro-isoquinoline- 1-carboxamide | 39.27 |
| 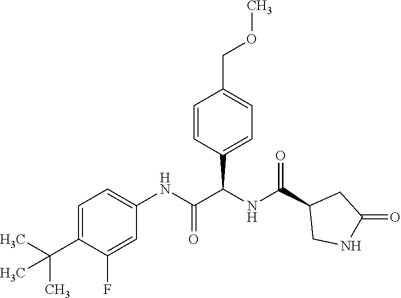 [SMILES: COCc1ccc([C@@H](NC(=O)[C@@H]2CNC(=O)C2)C(=O)Nc2ccc(C(C)(C)C)c(F)c2)cc1] | (3S)-N-((1R)-2-((4-tert-butyl-3- fluorophenyl)amino)-1-(4- (methoxymethyl)phenyl)-2-oxoethyl)-5- oxopyrrolidine-3-carboxamide | 38.73 |
| 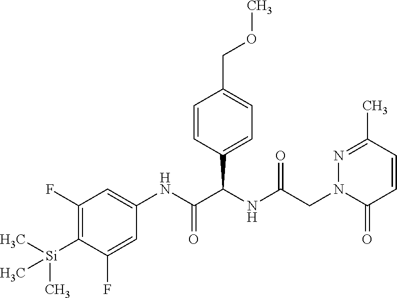 [SMILES: COCc1ccc([C@@H](NC(=O)Cn2nc(C)ccc2=O)C(=O)Nc2cc(F)c([Si](C)(C)C)c(F)c2)cc1] | (2R)-N-(3,5-difluoro-4- (trimethylsilyl)phenyl)-2-(4- (methoxymethyl)phenyl)-2-(((3-methyl-6- oxopyridazin-1(6H)- yl)acetyl)amino)acetamide | 38.70 |
| 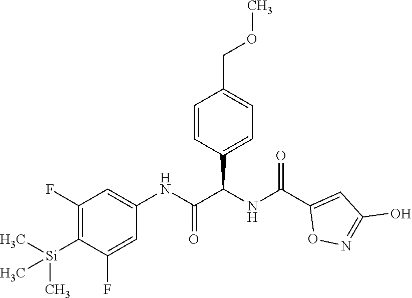 [SMILES: COCc1ccc([C@@H](NC(=O)c2cc(O)no2)C(=O)Nc2cc(F)c([Si](C)(C)C)c(F)c2)cc1] | N-((1R)-2-((3,5-difluoro-4- (trimethylsilyl)phenyl)amino)-1-(4- (methoxymethyl)phenyl)-2-oxoethyl)-3- hydroxy-N-methyl-1,2-oxazole-5- carboxamide | 37.44 |
| 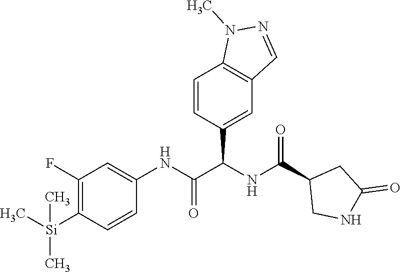 [SMILES: Cn1ncc2cc([C@@H](NC(=O)[C@@H]3CNC(=O)C3)C(=O)Nc3ccc([Si](C)(C)C)c(F)c3)ccc21] | (3S)-N-(2-((3-fluoro-4- (trimethylsilyl)phenyl)amino)-1-(1- methyl-1H-indazol-5-yl)-2-oxoethyl)-5- oxopyrrolidine-3-carboxamide | 36.72 |
| 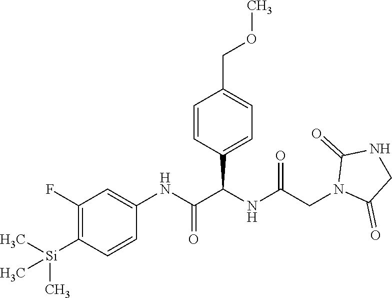 [SMILES: COCc1ccc([C@@H](NC(=O)CN2C(=O)CNC2=O)C(=O)Nc2ccc([Si](C)(C)C)c(F)c2)cc1] | (2R)-2-(((2,5-dioxoimidazolidin-1- yl)acetyl)amino)-N-(3-fluoro-4- (trimethylsilyl)phenyl)-2-(4- (methoxymethyl)phenyl)acetamide | 36.29 |
| 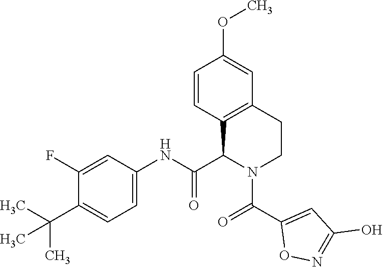 [SMILES: COc1ccc2c(c1)CCN(C(=O)c1cc(O)no1)[C@H]2C(=O)Nc1ccc(C(C)(C)C)c(F)c1] | N-(4-tert-butyl-3-fluorophenyl)-2-((3- hydroxy-1,2-oxazol-5-yl)carbonyl)-6- methoxy-1,2,3,4-tetrahydroisoquinoline- 1-carboxamide | 35.99 |
| 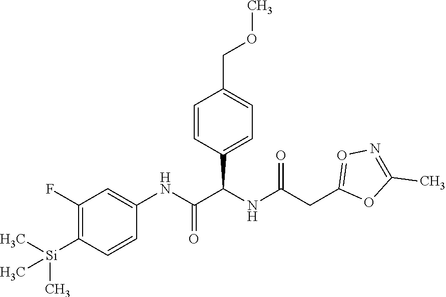 | (2R)-N-(3-fluoro-4- (trimethylsilyl)phenyl)-2-(4- (methoxymethyl)phenyl)-2-(((5-methyl- 1,3,4-oxadiazol-2- yl)acetyl)amino)acetamide | 35.55 |
| 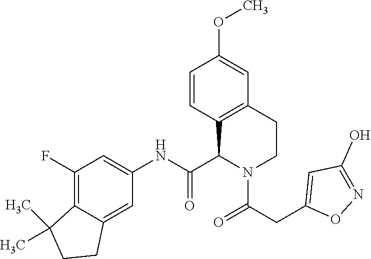 [SMILES: COc1ccc2c(c1)CCN(C(=O)Cc1cc(O)no1)[C@H]2C(=O)Nc1cc(F)c2c(c1)CCC2(C)C] | (1R)-N-(7-fluoro-1,1-dimethyl-2,3- dihydro-1H-inden-5-yl)-2-((3-hydroxy- 1,2-oxazol-5-yl)acetyl)-6-methoxy- 1,2,3,4-tetrahydroisoquinoline-1- carboxamide | 33.86 |
| 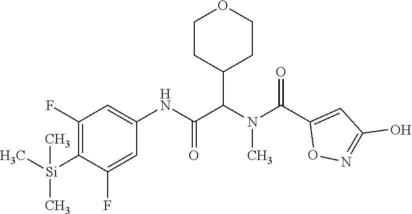 [SMILES: CN(C(=O)c1cc(O)no1)C(C(=O)Nc1cc(F)c([Si](C)(C)C)c(F)c1)C1CCOCC1] | N-(2-((3,5-difluoro-4- (trimethylsilyl)phenyl)amino)-2-oxo-1- (tetrahydro-2H-pyran-4-yl)ethyl)-3- hydroxy-N-methyl-1,2-oxazole-5- carboxamide | 31.71 |
| 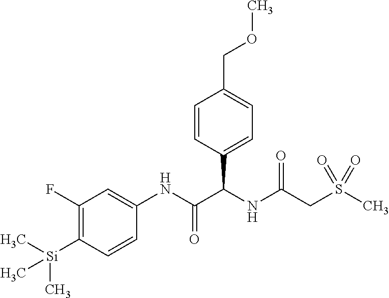 | (2R)-N-(3-fluoro-4- (trimethylsilyl)phenyl)-2-(4- (methoxymethyl)phenyl)-2- (((methylsulfonyl)acetyl)amino)acetamide | 31.30 |
| 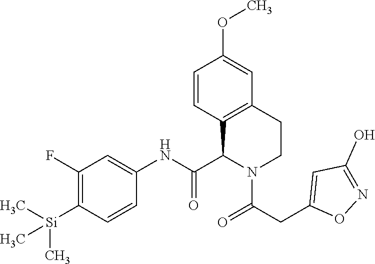 [SMILES: COc1ccc2c(c1)CCN(C(=O)Cc1cc(O)no1)[C@H]2C(=O)Nc1ccc([Si](C)(C)C)c(F)c1] | (1R)-N-(3-fluoro-4- (trimethylsilyl)phenyl)-2-((3-hydroxy- 1,2-oxazol-5-yl)acetyl)-6-methoxy- 1,2,3,4-tetrahydroisoquinoline-1- carboxamide | 31.20 |
| 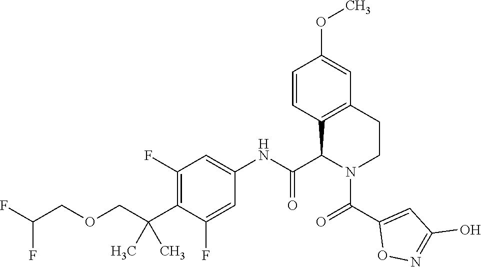 [SMILES: COc1ccc2c(c1)CCN(C(=O)c1cc(O)no1)[C@H]2C(=O)Nc1cc(F)c(C(C)(C)COCC(F)F)c(F)c1] | (1R)-N-(4-(1-(2,2-difluoroethoxy)-2- methylpropan-2-yl)-3,5-difluorophenyl)- 2-((3-hydroxy-1,2-oxazol-5-yl)carbonyl)- 6-methoxy-1,2,3,4- tetarhydroisoquinoline-1-carboxamide | 30.31 |
| 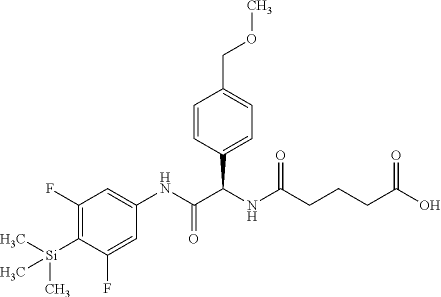 [SMILES: COCc1ccc([C@@H](NC(=O)CCCC(=O)O)C(=O)Nc2cc(F)c([Si](C)(C)C)c(F)c2)cc1] | 5-(((1R)-2-((3,5-difluoro-4- (trimethylsilyl)phenyl)amino)-1-(4- (methoxymethyl)phenyl)-2- oxoethyl)amino)-5-oxopentanoic acid | 26.72 |
| 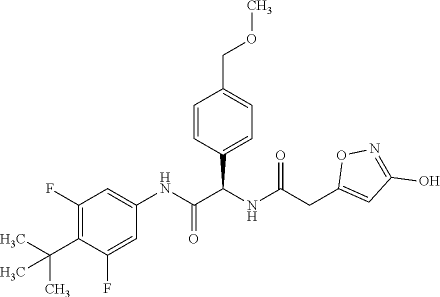 [SMILES: COCc1ccc([C@@H](NC(=O)Cc2cc(O)no2)C(=O)Nc2cc(F)c(C(C)(C)C)c(F)c2)cc1] | (2R)-N-(4-tert-butyl-3,5-difluorophenyl)- 2-(((3-hydroxy-1,2-oxazol-5- yl)acetyl)amino)-2-(4- (methoxymethyl)phenyl)acetamide | 26.27 |
| 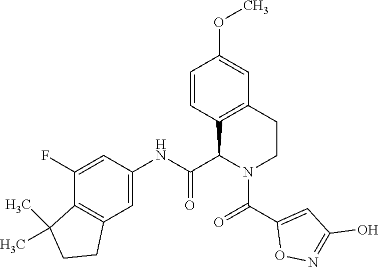 [SMILES: COc1ccc2c(c1)CCN(C(=O)c1cc(O)no1)[C@H]2C(=O)Nc1cc(F)c2c(c1)CCC2(C)C] | (5R)-N-(7-fluoro-1,1-dimethyl-2,3- dihydro-1H-inden-5-yl)-6-((3-hydroxy- 1,2-oxazol-5-yl)carbonyl)-2-methoxy- 5,6,7,8-tetrahydro-1,6-naphthyridine-5- carboxamide | 25.14 |
| 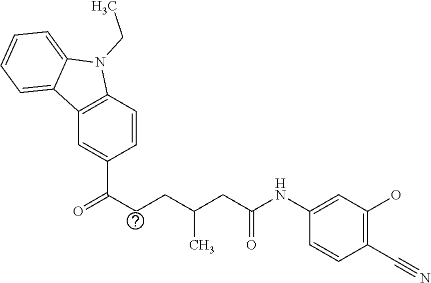 [SMILES: CCn1c2ccccc2c2cc(C(=O)CCC(C)CC(=O)Nc3ccc(C#N)c(O)c3)ccc21] | N-{4-[(3-chloro-4-cyanophenyl)amino]- 2-methyl-4-oxobutyl}-9-ethyl-9H- carbazole-3-carboxamide | 24.46 |
| 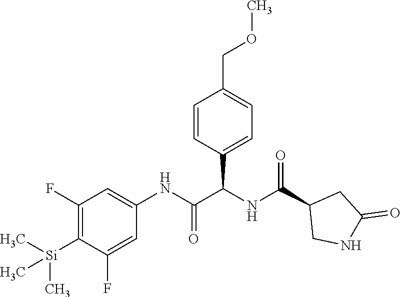 [SMILES: COCc1ccc([C@@H](NC(=O)[C@@H]2CNC(=O)C2)C(=O)Nc2cc(F)c([Si](C)(C)C)c(F)c2)cc1] | (3S)-N-((1R)-2-((3,5-difluoro-4- (trimethylsilyl)phenyl)amino)-1-(4- (methoxymethyl)phenyl)-2-oxoethyl)-5- oxopyrrolidine-3-carboxamide | 23.62 |
| 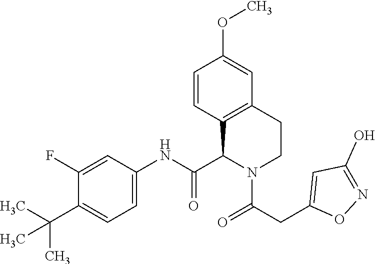 [SMILES: COc1ccc2c(c1)CCN(C(=O)Cc1cc(O)no1)[C@H]2C(=O)Nc1ccc(C(C)(C)C)c(F)c1] | (1R)-N-(4-tert-butyl-3-fluorophenyl)-2- ((3-hydroxy-1,2-oxazol-5-yl)acetyl)-6- methoxy-1,2,3,4-tetrahydroisoquinoline- 1-carboxamide | 23.61 |
| 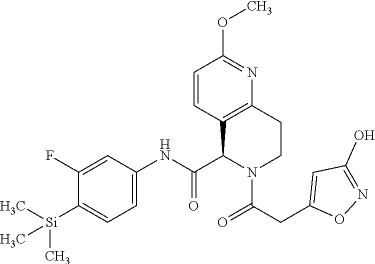 [SMILES: COc1ccc2c(n1)CCN(C(=O)Cc1cc(O)no1)[C@H]2C(=O)Nc1ccc([Si](C)(C)C)c(F)c1] | (5R)-N-(3-fluoro-4- (trimethylsilyl)phenyl)-6-((3-hydroxy- 1,2-oxazol-5-yl)acetyl)-2-methoxy- 5,6,7,8-tetrahydro-1,6-naphthyridine-5- carboxamide | 22.19 |
| 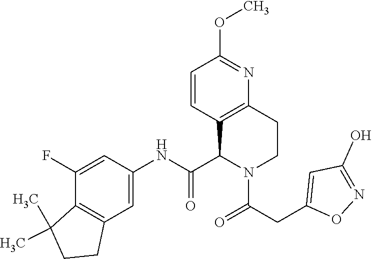 [SMILES: COc1ccc2c(n1)CCN(C(=O)Cc1cc(O)no1)[C@H]2C(=O)Nc1cc(F)c2c(c1)CCC2(C)C] | (5R)-N-(7-fluoro-1,1-dimethyl-2,3- dihydro-1H-inden-5-yl)-6-((3-hydroxy- 1,2-oxazol-5-yl)acetyl)-2-methoxy- 5,6,7,8-tetrahydro-1,6-naphthyridine-5- carboxamide | 19.25 |
| 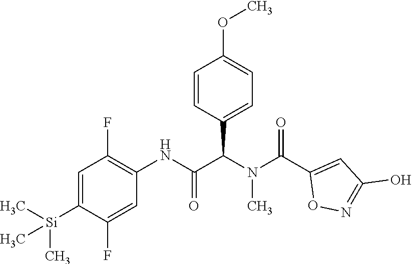 [SMILES: COc1ccc([C@H](C(=O)Nc2cc(F)c([Si](C)(C)C)cc2F)N(C)C(=O)c2cc(O)no2)cc1] | N-((1R)-2-((2,5-difluoro-4- (trimethylsilyl)phenyl)amino)-1-(4- methoxyphenyl)-2-oxoethyl)-3-hydroxy- N-methyl-1,2-oxazole-5-carboxamide | 18.17 |
| 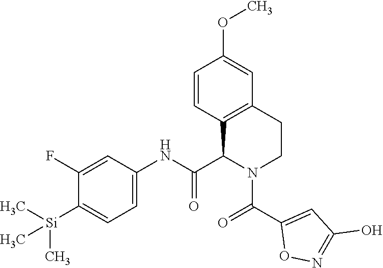 [SMILES: COc1ccc2c(c1)CCN(C(=O)c1cc(O)no1)[C@H]2C(=O)Nc1ccc([Si](C)(C)C)c(F)c1] | N-(3-fluoro-4-(trimethylsilyl)phenyl)-2- ((3-hydroxy-1,2-oxazol-5-yl)carbonyl)-6- methoxy-1,2,3,4-tetrahydroisoquinoline- 1-carboxamide | 7.69 |
| 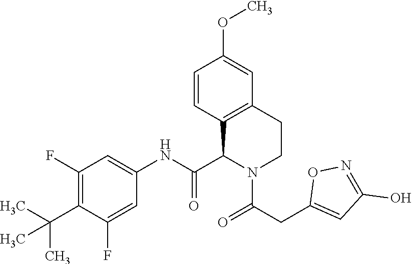 [SMILES: COc1ccc2c(c1)CCN(C(=O)Cc1cc(O)no1)[C@H]2C(=O)Nc1cc(F)c(C(C)(C)C)c(F)c1] | (1R)-N-(4-tert-butyl-3,5-difluorophenyl)- 2-((3-hydroxy-1,2-oxazol-5-yl)acetyl)-6- methoxy-1,2,3,4-tetrahydroisoquinoline- 1-carboxamide | −10.92 |
| 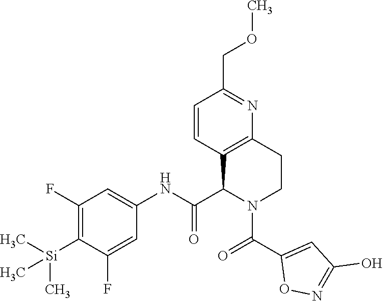 [SMILES: COCc1ccc2c(n1)CCN(C(=O)c1cc(O)no1)[C@H]2C(=O)Nc1cc(F)c([Si](C)(C)C)c(F)c1] | (5R)-N-(3,5-difluoro-4- (trimethylsilyl)phenyl)-6-((3-hydroxy- 1,2-oxazol-5-yl)carbonyl)-2- (methoxymethyl)-5,6,7,8-tetrahydro-1,6- naphthyridine-5-carboxamide | −15.53 |
| 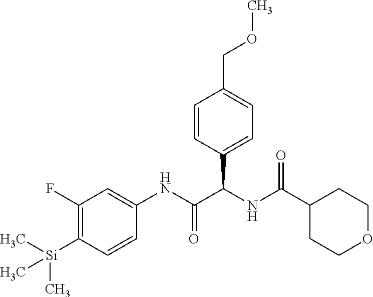 [SMILES: COCc1ccc([C@@H](NC(=O)C2CCOCC2)C(=O)Nc2ccc([Si](C)(C)C)c(F)c2)cc1] | N-((1R)-2-((3-fluoro-4- (trimethylsilyl)phenyl)amino)-1-(4- (methoxymethyl)phenyl)-2- oxoethyl)tetrahydro-2H-pyran-4- carboxamide | −20.07 |

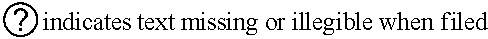

As shown in Table 1 above, the activity observed from sRICA showed no correlation with the compounds' inhibitory potencies or molecular weights.

Further studies were carried out with a representative compound (Compound 1) selected from Table 1 above to investigate the dose-dependent response of Compound 1 on skin samples treated to elicit a Th17 response generally following the same procedure. Like above, the study was normalized to the % max of IL17A production (i.e. the average IL17A concentration of Th17 activated skin alone) was set to 100%, all other values (untreated no activation and treated samples) were obtained as a percentage of this. The results are summarized below in Table 2.

TABLE 2

| | | Activity in sRICA |
|---|---|---|
| Concentration of | | IL17A (% of max. |
| Compound 1 | | induction) |
| 1 | nM | 94.03 |
| 3 | nM | 94.00 |
| 10 | nM | 71.95 |
| 30 | nM | 103.64 |
| 100 | nM | 49.97 |
| 300 | nM | 36.30 |
| l | μM | 25.45 |
| 3 | μM | 22.97 |

### EXAMPLE 3: Effect of Compound 1 on Induced Psoriasis in Mice

Topical application of imiquimod (IMQ), a TLR7/8 ligand and potent immune activator, is applied to the shaved backs and ears of mice to induce psoriasis. Information on the murine model of IMQ-induced psoriasis can be found at van der Fits L, Mourits S, Voerman JS, Kant M, Boon L, Laman JD, et al. Imiquimod-induced psoriasis-like skin inflammation in mice is mediated via the IL-23/IL-17 axis. J Immunol. 2009; 182:5836-45, which is hereby incorporated by reference in its entirety. Following administration of IMQ, mice ears and skin are observed for erythema, occurrence of scales and thickness after a period of six days. One group of mice were administered 0.05% topical clobetasol cream (10% body surface area), a potent topical steroid, as a positive control, and one group of mice received topical vehicle lacking Compoun 1 (10% body surface area), and one group was administered Compound 1 (3% formulation)(10% body surface area).

The mice were evaluated for erythema, presence of scales, thickness of ear tissue and body weight following administration of the treatments described above. Erythema and scales are determined by comparing the mice to a reference for color and severity of scaling. The results are summarized in Table 4.

TABLE 4

| | | IMQ + | p-value (IMQ + Vehicle |
|---|---|---|---|
| | IMQ + | Compound 1 + | vs. IMQ + |
| | Vehicle | Vehicle | Compound 1 + Vehicle) |
| Erythema (Score | 3.825 | 2.25 | ****p < 0.00001 |
| on PASI scale) | | | |
| Scales | 3.425 | 1.025 | ****p < 0.00001 |
| Left-ear thickness | 1.4 | 1.29 | ****p < 0.00001 |
| (fold-change) | | | |
| Body weight * | 98.50% | 100.50% | n.s. |
| (% of original) | | | |

As shown, mice treated with Compound 1 showed significant improvements in erythema and scales occurrence. Additionally, ear thickness in mice given Compound 1 showed

| | |
|---|---|
| 10 μM | 19.31 |

Statistical significance was established for all concentrations over 30 nM. These results establish that the IC50 of Compound 1 is approximately 100 nM.

### Example 2-Protein Expression Following Treatment with Rorγt INHIBITORS

Studies were carried out to determine the effect of two RORγt inhibitors of the present disclosure (i.e., Compound 1 and Compound 2, prepared in formulation as described in Example 1) on Th17 associated genes. Skin was harvested from subjects 24 hours after induction of inflammation. The RNA from the samples were isolated, and expression of the genes were quantified through real-time quantitative reverse transcription PCR. Clobetasol was used as a positive control. The results are summarized below in Table 3.

TABLE 3

| | Compound 1 | Compound 2 | Clobetasol |
|---|---|---|---|
| IL-17A | 43% reduction | 56% reduction | 68% reduction |
| TNFα | 47% reduction | 60% reduction | 25% reduction |
| IL-36γ | 7% reduction | 48% reduction | 60% reduction |
| CCL-20 | No change | 42% reduction | 10% reduction |
| S100A7 | 62% reduction | 60% reduction | 62% reduction |
| SerapinB | 50% reduction | 60% reduction | 65% reduction |
| IL-19 | 38% reduction | 29% reduction | 53% reduction |
| FoxP3 | 28-fold increase | 12-fold increase | 6-fold increase |

As shown in Table 2 above, there was a significant reduction in several Th17 associated genes, as well as an increase in FoxP3. The results show a reduction in the IL17A:FoxP3 ratio, which points toward re-polarization of the pathogenic Th17 response towards an anti-inflammatory tolerance response reduced inflammation. Mice administered clobetasol showed significant weight loss, signaling that it induced systemic toxicity.

What is claimed is:

1. A topical composition comprising a dermatologically acceptable excipient and a compound selected from the following:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide
N-(1-(4-methoxyphenyl)-2-oxo-2-((4-(trimethylsilyl)phenyl)amino)ethyl)—N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)-yl)acetyl)amino)acetamide;
N-(1-(4-methoxyphenyl)-2-oxo-2-((4-(trimethylsilyl)phenyl)amino)ethyl)—N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindo1-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)—N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N-(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)—N ′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3 S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)—N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)—N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide;
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)carbamoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)-yl)acetyl)amino)—N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-2-((4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)—N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-14-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide;
or a stereoisomer, tautomers, or pharmaceutically acceptable salts thereof.

2. The topical composition of claim 1, wherein the composition is in the form of a cream, a lotion, a foam, a gel, a spray or an ointment.

3. The topical composition of claim 1 or 2, wherein the compound of Formula (I) is at a concentration of about 0.001 wt. % to about 25 wt. %.

4. A method for treating an autoimmune disorder, the method comprising topically administering to a subject in need thereof an effective amount of a topical composition according to any of claims 1-3.

5. The method according to claim 4, wherein the autoimmune disorder is an autoimmune disorder of the skin.

6. The method according to claim 4 or 5, wherein the autoimmune disorder is consequent to dysfunction of Th17 cells or the release of IL-17 (e.g., IL-17A).

7. The method according to any of claims 4-6, wherein the autoimmune disorder is psoriasis, dermatitis, alopecia, cutaneous graft-versus-host-disease (cGVHD), ichthyosis, systemic sclerosis, vitiligo, rosacea, acne, urticaria, Behcet's disease, or lupus erythematosus.

8. The method according to any of claims 4-7, wherein the autoimmune disorder is psoriasis.

9. The method according to any of claims 4-8, wherein the autoimmune disorder is psoriasis vulgaris (i.e., plaque psoriasis), guttate psoriasis, inverse psoriasis (i.e., flexural psoriasis), pustular psoriasis (e.g., Von Zumbusch psoriasis, palmoplantar pustulosis, acropustulosis), psoriatic arthritis, erythrodermic psoriasis, nail psoriasis or scalp psoriasis.

10. The method according to any of claims 4-9, wherein the autoimmune disorder is psoriasis vulgaris (i.e., plaque psoriasis).

11. The method according to any of claims 4-7, wherein the autoimmune disorder is dermatitis.

12. The method according to any of claim 4-7 or 11, wherein the autoimmune disorder is atopic dermatitis (e.g., mild, moderate, or severe (including mild-to-moderate and moderate-to-severe) atopic dermatitis, Asian atopic dermatitis, European atopic dermatitis, pediatric atopic dermatitis), stasis dermatitis, contact dermatitis, dyshidriotic dermatitis, seborrheic dermatitis, neurodermatitis, nummular dermatitis, or stasis dermatitis.

13. The method according to any of claim 4-7 or 11-12, wherein the autoimmune disorder is atopic dermatitis (e.g., Asian atopic dermatitis, European atopic dermatitis).

14. The method according to any of claim 4-7 or 11-13, wherein the autoimmune disorder is Asian atopic dermatitis.

15. The method according to any of claims 4-7, wherein the autoimmune disorder is alopecia.

16. The method according to any of claim 4-7 or 15, wherein the autoimmune disorder is alopecia areata (e.g., alopecia totalis, alopecia universalis), cicatricial alopecia (e.g., frontal fibrosing alopecia, lichen planus (e.g., lichen planopilaris), folliculitis decalvans), telogen effluvium or anagen effluvium.

17. The method according to any of claim 4-7 or 15-16, wherein the autoimmune disorder is alopecia areata (e.g., alopecia totalis, alopecia universalis).

18. The method according to any of claims 4-7, wherein the autoimmune disorder is ichthyosis.

19. The method according to any of claim 4-7 or 18, wherein the autoimmune disorder is acquired ichthyosis, autosomal ichthyosis, autosomal recessive congenital ichthyosis (e.g. CIE or lamellar), Chanarin-Dorfman syndrome, CHILD syndrome, chondrodysplasia punctata syndrome, Darier disease, epidermal nevus syndrome, epidermolytic ichthyosis, erythrokeratodermias, Giroux-Barbeau syndrome, harlequin ichthyosis, Hailey-Hailey disease, ichthyosis en confetti, ichthyosis hystrix, ichthyosis vulgaris, keratosis follicularis spinulosa decalvans, keratitis-ichthyosis-deafness syndrome, multiple sulfatase deficiency, netherton syndrome, pachyonychia congenita, palmoplantar keratoderma, peeling skin syndrome, pityriasis rubra pilaris, Refsum disease, Rud's syndrome, Sjögren-Larsson syndrome, trichiothiodystrophy, or X-linked ichthyosis.

20. The method according to any of claims 4-7, wherein the autoimmune disorder is systemic sclerosis.

21. The method according to any of claim 4-7 or 20, wherein the autoimmune disorder is morphea, linear scleroderma, limited systemic scleroderma, diffuse systemic scleroderma, CREST syndrome, sine sclerosis.

22. The method according to any of claims 4-7, wherein the autoimmune disorder is vitiligo.

23. The method according to any of claim 4-7 or 22, wherein the autoimmune disorder is vitiligo (e.g., non-segmental vitiligo or segmental vitiligo).

24. The method according to any of claims 4-7, wherein the autoimmune disorder is rosacea.

25. The method according to any of claim 4-7 or 24, wherein the autoimmune disorder is erythematotelangiectatic rosacea, papulopustular rosacea (e.g. acne), rhinophyma, or ocular rosacea.

26. The method according to any of claims 4-7, wherein the autoimmune disorder is uticaria.

27. The method according to any of claim 4-7 or 26, wherein the autoimmune disorder is chronic spontaneous uticaria.

28. The method according to any of claims 4-7, wherein the autoimmune disorder is Behcet's disease.

29. The method according to any of claims 4-7, wherein the autoimmune disorder is lupus erythematosus.

30. The method according to any of claim 4-7 or 29, wherein the autoimmune disorder is systemic lupus erythematosus, discoid lupus erythematosus, or drug-induced lupus.

31. The method according to any of claims 4-7, wherein the autoimmune disorder is cutaneous graft-versus-host-disease (cGVHD).

32. A method for method for reducing inflammation in mammalian skin, the method comprising topically administering to the mammalian skin an effective amount of a topical composition of any of claims 1-3.

33. A method for reducing the release of an inflammatory biomarker (e.g., IL-17, IL-22), or reducing the incidence of skin lesions on a subject in need thereof, the method comprising topically administering to the mammalian skin a therapeutically effective amount of a topical composition of any of claims 1-3.

34. The method of claim 32 or 33, wherein the mammalian skin comprises human skin.
